(19) United States
(12) Patent Application Publication
(10) Pub. No.: US 2005/0124667 A1
(43) Pub. Date: Jun. 9, 2005

(54) Oxamide inhibitors of plasminogen activator inhibitor-1

(76) Inventors: Eric Sartori, Paris (FR); Magali Maillet, Suresne (FR); Marie France Paugam, Le Chesnay (FR); Eric Nicolai, Rueil-Malmaison (FR); Michael Lawrence, Yardley, PA (US); Karnail S. Atwal, Pennington, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/969,480

(22) Filed: Oct. 20, 2004

Related U.S. Application Data
(60) Provisional application No. 60/515,895, filed on Oct. 30, 2003.

(51) Int. Cl. A61K 31/455; A61K 31/4172; A61K 31/277; A61K 31/325; A61K 31/165

(52) U.S. Cl. 514/355; 514/486; 514/522; 514/400; 514/599; 514/616; 514/534

(57) ABSTRACT

Methods of treating disorders associated with elevated levels of PAI-1 are disclosed comprising administering to a patient in need thereof a therapeutically effective amount of at least one compound of formula at least one compound of formula (I), 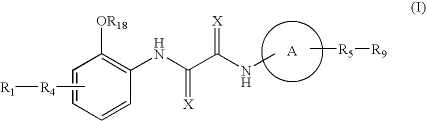 or a pharmaceutically-acceptable salt, prodrug, stereoismer or solvate thereof, wherein:
A is aryl or heteroaryl, X is O or S, and R₁-R₉ and R₁₈ are defined herein. The invention also pertains to pharmaceutical compositions and compounds within the scope of formula (I) as well as medicaments and articles of manufacture comprising compounds of formula (I).

This application claims the benefit of priority from U.S. Provisional Application Ser. No. 60/515,895, filed Oct. 30, 2003.

### FIELD OF THE INVENTION

This invention relates to methods of using oxamide compounds to inhibit Plasminogen Activator Inhibitor-1 (“PAI-1”) to treat disorders associated with elevated levels of PAI-1, as well as pharmaceutical compositions and articles of manufacture comprising the oxamide compounds.

### BACKGROUND OF THE INVENTION

Plasminogen activator inhibitor-1 (“PAI-1”) is a member of the serine protease inhibitor (SERPIN) superfamily of proteins and plays a major role in the regulation of the plasminogen-plasmin system. PAI-1 is known to be the principal inhibitor of the serine proteases tissue-type plasminogen activator (t-PA) and urokinase-type plasminogen activator (u-PA). In the plasma, t-PA cleaves the zymogen plasminogen to the active enzyme plasmin, which can degrade fibrin clots (fibrinolysis or thrombolysis) thereby exerting an antithrombotic effect. Therefore, through the regulation of t-PA, PAI-1 plays an important role in hemostasis. An increased level of PAI-1 is believed to be a risk factor in thrombotic conditions such as venous thrombosis, atherosclerosis, and arterial thrombosis, which can result in deep vein thrombosis, pulmonary embolism, myocardial infarction, stroke, etc. See e.g., Wu, Current Drug Targets, 2, 27, (2002); Dawson, et al., Atherosclerosis, 95, 105 (1992); Wiman, et al., Thrombosis and Haemostasis, 74, 71, (1995); V. Salomaa, et al., Circulation, 91, 284 (1995); and Eitzman, Blood, 96, 4212 (2000). In animal experiments, inhibitors of PAI-1 activity have been shown to be effective at treating thrombotic conditions. See e.g., Berry, et al, British Journal of Pharmacology, 125, 29 (1998) and Friedrich, et al., Circulation, 96, 916, (1997).

In tissue matrices, u-PA converts plasminogen to plasmin which activates matrix metalloproteases (MMPs) that degrade extracellular matrix (ECM). Through this regulation of u-PA, PAI-1 therefore plays an important role in cellular migration and tissue remodelling processes. PAI-1 is thus believed to modulate diseases and conditions such as wound healing, angiogenesis, cancer invasion, and metastasis. Increased levels of PAI-1 have been associated with poor prognosis in cancer patients (T. L. Frandesen, Drugs Future, 873, 1998; Pappot, et al, Biol. Chem. Hoppe-Seyler, 376, 259, 1995). PAI-1 has additionally been associated with other conditions and diseases such as obesity and insulin resistance (Juhan-Vague, et al, Journal of Thrombosis and Haemostasis, 1, 1575, 2003), inflammatory diseases, such as asthma (Cho, et al, Journal of Allergy and Clinical Immunology, 108, 212, 2001), and renal disease (Brown, et al, Journal of Nephrology, 15, 230, 2002). See also Tsikouris et al., J. Clin. Pharmacol., 42:1187, 2002 and Binder et al., News Physiol, Sci, 17, 56, 2002.

Accordingly, compounds that inhibit PAI-1 would be useful in the treatment of several disease states and disorders, especially thromboembolic disorders.

### SUMMARY OF THE INVENTION

The instant invention pertains to methods of inhibiting PAI-1 inhibitors Comprising administering to a patient in need thereof a therapeutically effective amount of at least one compound of formula (I), 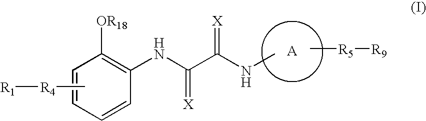
or a pharmaceutically-acceptable salt, prodrug, stereoismer or solvate thereof, wherein:
A is aryl or heteroaryl;
X is O or S;
R₁-R₉ are independently selected from hydrogen, alkyl, substituted alkyl, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀, —N₁₃C(═O)NR₁₁R₁₂, halogen, nitro and cyano;
or any two of R₁-R₉ located on neighboring carbon atoms of the phenyl ring may be taken together to form a fused ring system in combination with the phenyl ring to which they are attached, wherein the fused ring system may be optionally further substituted;
R₁₀, R₁₁, R₁₂ and R₁₃ are independently selected from hydrogen, alkyl, substituted alkyl, aryl, heteroaryl, cycloalkyl and heterocyclo, wherein each instance of R₁₀, R₁₁, R₁₂ and/or R₁₃ is selected independently; and
R₁₈ is hydrogen, alkyl or substituted alkyl (preferably hydrogen, methyl or CF₃, more preferably hydrogen).

The invention further relates to compounds having formula (Ia), 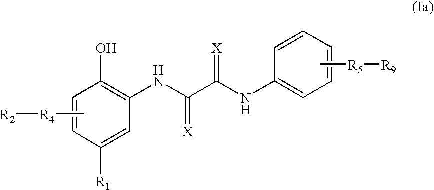
or a pharmaceutically-acceptable salt, prodrug, stereoisomer or solvate thereof, wherein:
X is O or S:
R₁ is halogen; and
R₅ is —OR₁₇ or —SR₁₇.
R₂-R₄ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
R₆-R₉ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
or any two of R₆-R₉ located on neighboring carbon atoms of the phenyl ring may be taken together to form a fused ring system in combination with the phenyl ring, wherein the fused ring system may be optionally further substituted; and
R₁₇ is hydrogen, alkyl, substituted alkyl, cycloalkyl, aryl, heteroaryl or heterocyclo.

### DESCRIPTION OF THE INVENTION

Listed below are definitions of various terms used to describe this invention. These definitions apply to the terms as they are used throughout this specification, unless otherwise limited in specific instances, either individually or as part of a larger group.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms. When a subscript is used with reference to an alkyl or other group, the subscript refers to the number of carbon atoms that the group may contain. For example, the term “C₀₋₄alkyl” includes a bond and alkyl groups of 1 to 4 carbon atoms.

The term “substituted alkyl” refers to an alkyl group substituted by one to four substituents selected from halogen, hydroxy, alkoxy, keto (═O), alkanoyl, aryloxy, alkanoyloxy, NRₐR_{b}, alkanoylamino, aroylamino, aralkanoylamino, substituted alkanoylamino, substituted arylamino, substituted aralkanoylamino, thiol, alkylthio, arylthio, aralkylthio, alkylthiono, arylthiono, aralkylthiono, alkylsulfonyl, arylsulfonyl, aralkylsulfonyl, —SO₂NRₐR_{b}, nitro, cyano, —CO₂H, —CONRₐR_{b}, alkoxycarbonyl, aryl, guanidino and heteroaryls or heterocyclos (such as indolyl, imidazolyl, furyl, thienyl, thiazolyl, pyrrolidyl, pyridyl, pyrimidyl and the like), wherein Rₐ and R_{b} ate selected from hydrogen, alkyl, aryl, aralkyl, cycloalkyl, cycloalkylalkyl, heteroaryl, heteroarylalkyl, heterocycle, and heterocyclealkyl. The substituent on the alkyl optionally in turn may be further substituted, in which case it will be with substituted one or more of C₁₋₄alkyl, C₂₋₄alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenyloxy, and/or benzyloxy.

The term “alkenyl” refers to straight or branched chain hydrocarbon groups of 2 to 20 carbon atoms, preferably 2 to 15 carbon atoms, and most preferably 2 to 8 carbon atoms, having at least one double bond, and depending on the number of carbon atoms, up to four double bonds.

The term “substituted alkenyl” refers to an alkenyl group substituted by one to two substituents selected from those recited above for substituted alkyl groups.

The term “alkynyl” refers to straight or branched chain hydrocarbon groups of 2 to 20 carbon atoms, preferably 2 to 15 carbon atoms, and most preferably 2 to 8 carbon atoms, having at least one triple bond, and depending on the number of carbon atoms, up to four triple bonds.

The term “substituted alkynyl” refers to an alkynyl group substituted by one to two substituents selected from those recited above for alkyl groups.

When the term alkyl is used in connection with another group, as in heterocycloalkyl or cycloalkylalkyl, this means the identified (first named) group is bonded directly through an alkyl group which may be branched or straight chain (e.g., cyclopropylC₁₋₄alkyl means a cyclopropyl group bonded through a straight or branched chain alkyl group having one to four carbon atoms). In the case of substituents, as in “substituted cycloalkylalkyl,” the alkyl portion of the group, besides being branched or straight chain, may be substituted as recited above for substituted alkyl groups and/or the first named group (e.g., cycloalkyl) may be substituted as recited herein for that group.

The term “halogen” or “halo” refers to fluorine, chlorine, bromine and iodine.

The term “aryl” refers to monocyclic or bicyclic aromatic substituted or unsubstituted hydrocarbon groups having 6 to 12 carbon atoms in the ring portion, such as phenyl, naphthyl, and biphenyl groups. Each ring of the aryl may be optionally substituted with one to three R_{c} groups, wherein R_{c} at each occurrence is selected from alkyl, substituted alkyl, halogen, trifluoromethoxy, trifluoromethyl, —SR, —OR, —NRR′, —NRSO₂R′, —SO₂R, —SO₂NRR′, —CO₂R′, —C(═O)R′, —C(═O)NRR′, —OC(═O)R′, —OC(═O)NRR′, —NRC(═O)R′, —NRCO₂R′, phenyl, C₃₋₇ cycloalkyl, and five-to-six membered heterocyclo or heteroaryl, wherein each R and R′ is selected from hydrogen, alkyl, substituted alkyl, alkenyl, substituted alkenyl, phenyl, C₃₋₇cycloalkyl, and five-to-six membered heterocyclo or heteroaryl, except in the case of a sulfonyl group, then R is not going to be hydrogen. Each substituent R_{c} optionally in turn may be further substituted by one or more (preferably 0 to 2) R_{d} groups, wherein R_{d} is selected from C₁₋₆alkyl, C₂₋₆alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenylethyl, phenyloxy, and benzyloxy.

The term “aralkyl” refers to an aryl group bonded directly through an alkyl group, such as benzyl, wherein the alkyl group may be branched or straight chain. In the case of a “substituted aralkyl,” the alkyl portion of the group besides being branched or straight chain, may be substituted as recited above for substituted alkyl groups and/or the aryl portion may be substituted as recited herein for aryl. Thus, the term “optionally substituted benzyl” refers to the group 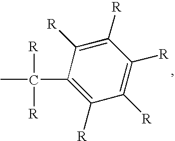
wherein each R group may be hydrogen or may also be selected from R_{c} as defined above, in turn optionally substituted with one or more R_{d}. At least two of these “R” groups should be hydrogen and preferably at least five of the “R” groups is hydrogen. A preferred benzyl group involves the alkyl-portion being branched to define

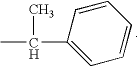

The term “heteroaryl” refers to a substituted or unsubstituted aromatic group for example, which is a 4 to 7 membered monocyclic, 7 to 11 membered bicyclic, or 10 to 15 membered tricyclic ring system, which has at least one heteroatom and at least one carbon atom-containing ring. Each ring of the heteroaryl group containing a heteroatom can contain one or two oxygen or sulfur atoms and/or from one to four nitrogen atoms, provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. It may optionally be substituted with one to three (preferably 0 to 2) R_{c} groups, as defined above for aryl, which in turn may be substituted with one or more (preferably o to 2) R_{d} groups, also as recited above.

Exemplary monocyclic heteroaryl groups include pyrrolyl, pyrazolyl, pyrazolinyl, imidazolyl, oxazolyl, isoxazolyl, thiazolyl 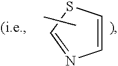
thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

The term “cycloalkyl” refers to a saturated or partially unsaturated non-aromatic cyclic hydrocarbon ring system, preferably containing 1 to 3 rings and 3 to 7 carbon atoms per ring, which may be substituted or unsubstituted and/or which may be fused with a C₃-C₇ carbocylic ring, a heterocyclic ring, or which may have a bridge of 3 to 4 carbon atoms. The cycloalkyl groups including any available carbon or nitrogen atoms on any fused or bridged rings optionally may have 0 to 3 (preferably 0-2) substituents selected from R_{c} groups, as recited above, and/or from keto (where appropriate) which in turn may be substituted with one to three R_{d} groups, also as recited above. Thus, when it is stated that a carbon-carbon bridge may be optionally substituted, it is meant that the carbon atoms in the bridged ring optionally may be substituted with an R_{c} group, which preferably is seleted from C₁₋₄alkyl, C₂₋₄alkenyl, halogen, haloalkyl, haloalkoxy, cyano, amino, C₁₋₄alkylamino, aminoC₁₋₄alkyl, hydroxy, hydroxyC₁₋₄alkyl, and C₁₋₄alkoxy. Exemplary groups include cyclopropyl, cyclobutyl, cyclopentyl, cyclohexyl, cycloheptyl, bicycloheptane, cycloctyl, cyclodecyl, cyclododecyl, and adamantyl.

The terms “heterocycle”, “heterocyclic” and “heterocyclo” each refer to a fully saturated or partially unsaturated nonaromatic cyclic group, which may be substituted or unsubstituted, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen, oxygen, and sulfur atoms, where the nitrogen and sulfur heteroatoms also optionally may be oxidized and the nitrogen heteroatoms also optionally may be quaternized. Preferably two adjacent heteroatoms are not simultaneously selected from oxygen and nitrogen. The heterocyclic group may be attached at any nitrogen or carbon atom. The heterocyclo groups optionally may have 0 to 3 (preferably 0-2) substituents selected from keto (═O), and/or one or more R_{c} groups, as recited above, which in turn may be substituted with one to three R_{d} groups, also as recited above.

Exemplary monocyclic heterocyclic groups include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic hetrocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Also included are smaller heterocyclos, such as epoxides and aziridines.

Unless otherwise indicated, when reference is made to a specifically-named aryl (e.g., phenyl), cycloalkyl (e.g., cyclohexyl), heterocyclo (e.g., pyrrolidinyl) or heteroaryl (e.g., indolyl), the reference is intended to include rings having 0 to 3, preferably 0-2, substituents selected from those recited above for the the aryl, cycloalkyl, heterocyclo and/or heteroaryl groups, as appropriate. Additionally, when reference is made to a specific heteroaryl or heterocyclo group, the reference is intended to include those systems having the maximum number of non-cumulative double bonds or less than the maximum number of double bonds. Thus, for example, the term “isoquinoline” refers to isoquinoline and tetrahydroisoquinoline.

Additionally, it should be understood that one skilled in the field may make appropriate selections for the substituents for the aryl, cycloalkyl, heterocyclo, and heteroaryl groups to provide stable compounds and compounds useful as pharmaceutically-acceptable compounds and/or intermediate compounds useful in making pharmaceutically-acceptable compounds. Thus, for example, in compounds of formula (I), when a substituent is a cyclopropyl ring, preferably the ring has no more than two substituents, and preferably said substituents do not comprise nitro (NO₂), more than one cyano group, or three halogen groups. Similarly, when m is 3, preferably R₆, the substituents on the phenyl ring A, are not all nitro, and so forth.

The term “heteroatoms” shall include oxygen, sulfur and nitrogen.

The term “haloalkyl” means an alkyl having one or more halo substituents.

The term “perfluoromethyl” means a methyl group substituted by one, two, or three fluoro atoms, i.e., CH₂F, CHF₂ and CF₃. The term “perfluoroalkyl” means an alkyl group having from one to five fluoro atoms, such as pentafluoroethyl.

The term “haloalkoxy” means an alkoxy group having one or more halo substituents. For example, “haloalkoxy” includes —OCF₃.

The term “carbocyclic” means a saturated or unsaturated monocyclic or bicyclic ring in which all atoms of all rings are carbon. Thus, the term includes cycloalkyl and aryl rings. The carbocyclic ring may be substituted in which case the substituents are selected from those recited above for cycloalkyl and aryl groups.

When the term “unsaturated” is used herein to refer to a ring or group, the ring or group may be fully unsaturated or partially unsaturated.

Definitions for the various other groups that are recited above in connection with substituted alkyl, substituted alkenyl, aryl, cycloalkyl, and so forth, are as follows: alkoxy is —OR^{e}, alkanoyl is —C(═O)R^{e}, aryloxy is —OAr, alkanoyloxy is —OC(═O)R^{e}, amino is —NH₂, alkylamino is —NHR^{e} or —N(R^{e})₂, arylamino is —NHAr or —NR^{e}Ar, aralkylamino is —NH—R^{f}-Ar, alkanoylamino is —NH—C(═O)R^{e}, aroylamino is —NH—C(═O)Ar, aralkanoylamino is —NH—C(═O)R^{f}-Ar, thiol is —SH, alkylthio is —SR^{e}, arylthio is —SAr, aralkylthio is —S—R^{f}-Ar, alkylthiono is —S(═O)R^{e}, arylthiono is —S(═O)Ar, aralkylthiono is —S(═O)R^{f}-Ar, alkylsulfonyl is —SO_{(q)}R^{e}, arylsulfonyl is —SO_{(q)}Ar, arylsulfonylamine is —NHSO_{(q)}Ar, alkylsulfonylamine is —NHSO₂R^{e}, aralkylsulfonyl is —SO_{(q)}R^{f}Ar, sulfonamido is —SO₂NH₂, substituted sulfonamide is —SO₂NHR^{e} or —SO₂N(R^{e})₂, nitro is —NO₂, carboxy is —CO₂H, carbamyl is —CONH₂, substituted carbamyl is —C(═O)NHR^{g} or —C(═O)NR^{g}R^{h}, alkoxycarbonyl is —C(═O)OR^{e}, carboxyalkyl is —R^{f}—CO₂H, sulfonic acid is —SO₃H, arylsulfonylamine is —NHSO_{(q)}Ar, guanidino is 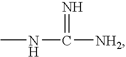
and ureido is 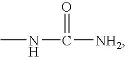
wherein R^{e} is alkyl or substituted alkyl as defined above, R^{f} is alkylene or substituted alkylene as defined above, R^{g} and R^{h} are selected from alkyl, substituted alkyl, aryl, aralkyl, cycloalkyl, heterocyclo, and heteraryl; Ar is an aryl as defined above, and q is 2 or 3.

Throughout the specification, groups and substituents thereof may be chosen by one skilled in the field to provide stable moieties and compounds.

The compounds of Formula (I) may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of Formula (I) may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for Formula (I) may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art. Salt forms of the compounds may be advantageous for improving the compound dissolution rate and oral bioavailability.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

Compounds of the Formula (I) may also have prodrug forms. Any compound that will be converted in vivo to provide the bioactive agent (i.e., the compound for formula I) is a prodrug within the scope and spirit of the invention.

Various forms of prodrugs are well known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Acamedic Press, 1985);
b) A Textbook of Drug Design and Development, edited by Krosgaard-Larsen and H. Bundgaard, Chapter 5, “Design and Application of Prodrugs,” by H. Bundgaard, p. 113-191 (1991); and
c) H. Bundgaard, Advanced Drug Delivery Reviews, 8, 1-38 (1992), each of which is incorporated herein by reference.

It should further be understood that solvates (e.g., hydrates) of the compounds of Formula (I) are also with the scope of the present invention. Methods of solvation are generally known in the art.

### Preferred Methods and Compounds

Preferred methods of treating a disorder associated with high levels of PAI-1 are those comprising administering to a patient in need of such treatment an effective amount of at least one compound having the formula (Ia), 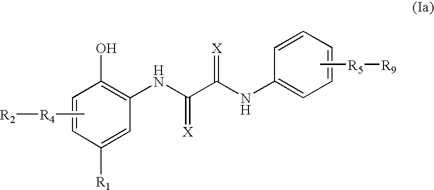
or a pharmaceutically-acceptable salt, prodrug, stereoisomer or solvate thereof, wherein:
X is O or S;
R₁ is halogen (especially chloro); and
R₅ is —OR₁₇ or —SR₁₇.
R₂-R₄ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
R₆-R₉ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
or any two of R₆-R₉ located on neighboring carbon atoms of the phenyl ring may be taken together to form a fused ring system in combination with the phenyl ring, wherein the fused ring system may be optionally further substituted; and
R₁₇ is hydrogen, alkyl, substituted alkyl, cycloalkyl, aryl heteroaryl or heterocyclo (especially hydrogen or C₁₋₆alkyl).

More preferred methods of treating a disorder associated with high levels of PAI-1 are those comprising administering to a patient in need of such treatment an effective amount of at least one compound within the scope of formula (Ia) in which at least one, and preferably all of the variables are chosen from:
R₂-R₄ and R₆-R₉ are independently selected from hydrogen, alkyl, substituted alkyl, halogen, nitro, cyano, —OR₁₀, —OC(═O)R₁₀, —CO₂R₁₀, —C(═O)NR₁₁R₁₂, —NR₁₁R₁₂, —S(═O)R₁₀, —SO₂R₁₀, and —SO₂NR₁₁R₁₂;
R₁₀ is hydrogen, C₁₋₆alkyl, substituted C₁₋₆alkyl, aryl or heteroaryl; and
R₁₁ and R₁₂ are selected from hydrogen, C₁₋₆alkyl and substituted C₁₋₆alkyl,
especially wherein the substituted C₁₋₆alkyl of groups R₁₀, R₁₁ and R₁₂ is substituted by one or more groups selected from
   (a) —C(═O)R₁₄, —CO₂R₁₄, —NR₁₅R₁₆; or
   (b) alkyl, aryl, heterocyclo or heteroaryl, each group of which may optionally be further substituted by C₁₋₆alkyl, C₁₋₆alkyloxy, C₁₋₆haloalkyloxy, halogen, nitro and cyano; and
R₁₄, R₁₅, and R₁₆ are independently selected from hydrogen, alkyl, substituted alkyl, aryl, heteroaryl, cycloalkyl and heterocyclo, each group of which may optionally be further substituted by halogen, nitro, cyano, C₁₋₆alkyl, C₁₋₆haloalkyl, C₁₋₆alkyloxy, and C₁₋₆haloalkyloxy (particularly where R₁₄ is C₁₋₆alkyl; and R₁₅, and R₁₆ are selected from hydrogen and C₁₋₆alkyl).

Preferred compounds within the scope of formula I(a), shown above, are those compounds, or a pharmaceutically-acceptable salt, prodrug, stereoisomer or solvate thereof, in which the variables are chosen from the following:
X is O or S;
R₁ is halogen (especially chloro); and
R₅ is —OR₁₇ or —SR₁₇.
R₂-R₄ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
R₆-R₉ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
or any two of R₆-R₉ located on neighboring carbon atoms of the phenyl ring may be taken together to form a fused ring system in combination with the phenyl ring, wherein the fused ring system may be optionally further substituted; and
R₁₇ is hydrogen, alkyl, substituted alkyl, cycloalkyl, aryl heteroaryl or heterocyclo (especially hydrogen or C₁₋₆alkyl).

More preferred compounds within the scope of formula (Ia), are those compounds, pharmaceutically-acceptable salt, prodrug, stereoisomer or solvate thereof, in which at least one, and preferably all of the variables are chosen from the following:
R₂-R₄ and R₆-R₉ are independently selected from hydrogen, alkyl, substituted alkyl, halogen, nitro, cyano, —OR₁₀, —OC(═O)R₁₀, —CO₂R₁₀, —C(═O)NR₁₁R₁₂, —NR₁₁R₁₂, —S(═O)R₁₀, —SO₂R₁₀, and —SO₂NR₁₁R₁₂;
R₁₀ is hydrogen, C₁₋₆alkyl, substituted C₁₋₆alkyl, aryl or heteroaryl; and
R₁₁ and R₁₂ are selected from hydrogen, C₁₋₆alkyl and substituted C₁₋₆alkyl,
especially wherein the substituted C₁₋₆alkyl of groups R₁₀, R₁ₜ and R₁₂ is substituted by one or more groups selected from
   (a) —C(═O)R₁₄, —CO₂R₁₄, —NR₁₅R₁₆; or
   (b) alkyl, aryl, heterocyclo or heteroaryl, each group of which may optionally be further substituted by C₁₋₆alkyl, C₁₋₆alkyloxy, C₁₋₆haloalkyloxy, halogen, nitro and cyano; and
R₁₄, R₁₅, and R₁₆ are independently selected from hydrogen, alkyl, substituted alkyl, aryl, heteroaryl, cycloalkyl and heterocyclo, each group of which may optionally be further substituted by halogen, nitro, cyano, C₁₋₆alkyl, C₁₋₆haloalkyl, C₁₋₆alkyloxy, and C₁₋₆haloalkyloxy (more especially where R₁₄ is C₁₋₆alkyl; and R₁₅, and R₁₆ are selected from hydrogen and C₁₋₆alkyl).

Even more preferred compounds within the scope of formula (Ia) are those having formula (Ib), 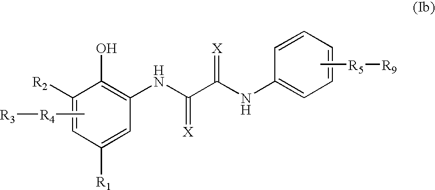
or a pharmaceutically-acceptable salt, prodrug, stereoisomer or solvate thereof, in which the variables are selected according to above described preferred compounds in the scope of formula (Ia). Especially preferred compounds within the scope of formula (Ib) are those compounds wherein R₂ is a halogen (especially chloro).

### Methods of Preparation

The compounds of the present invention may be synthesized by many methods available to those skilled in the art of organic chemistry. General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds. Examples of compounds of the present invention (where A=aryl) are exemplified in the general schemes below. One of skill understands that these schemes may be applied to intermediates and compounds in which A=heteroaryl.

Derivatives of formula (I) can be obtained through condensation of the appropriate N-phenyl-oxalamic acid ethyl ester with anilines or aromatic amines in various solvent, such as refuxing dioxane or xylene:

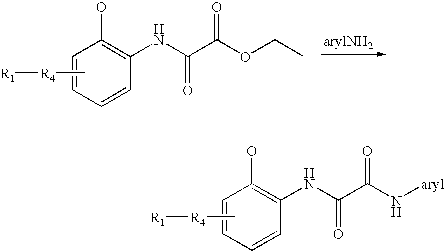

Alternatively, derivatives of formula (I) can be obtained through condensation of appropriately substituted benzo[1,4]oxazine-2,3-diones with anilines or aromatic amines in various solvent, such as refuxing tetrahydrofurane, dioxane or xylene:

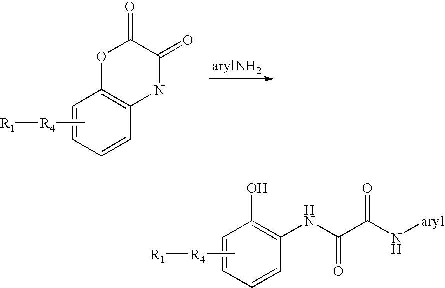

Alternatively, derivatives of formula (I) can be obtained through condensation of N-Aryl oxalamic ethyl esters with substituted amino phenols in various solvent, such as refuxing dioxane or xylene:

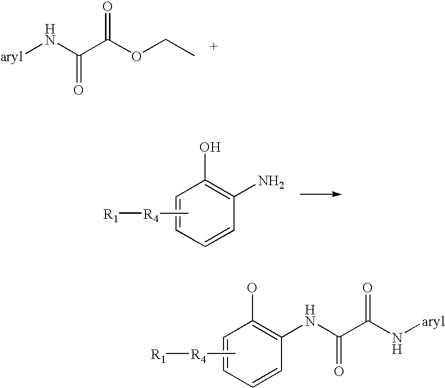

Symmetrical derivatives of formula (I) can be obtained through condensation of substituted anilines with dioxalyl chloride: 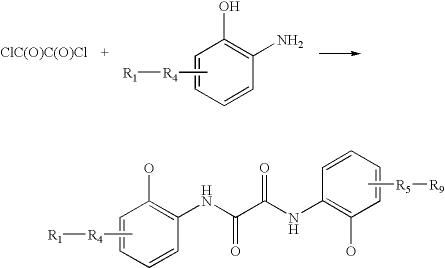
where R₁-R₄=R₅-R₉

Compounds for which R₂═NH₂ can be obtained by reduction of the corresponding nitro compounds using hydrogen and a catalyst or any other usual reducing method (Fe, HCl or SnCl2 etc)

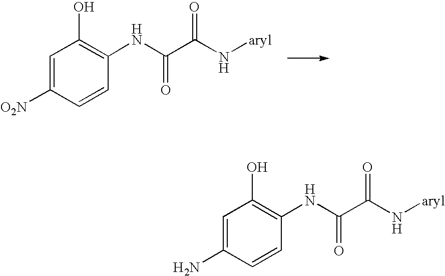

Compounds for which Ar is a phenol substituted by CONHR, in position para or meta to the phenol hydroxyl, are obtained through the coupling of the corresponding acids with amines using EDCI and HOBt as coupling reagents, or any other usual coupling method

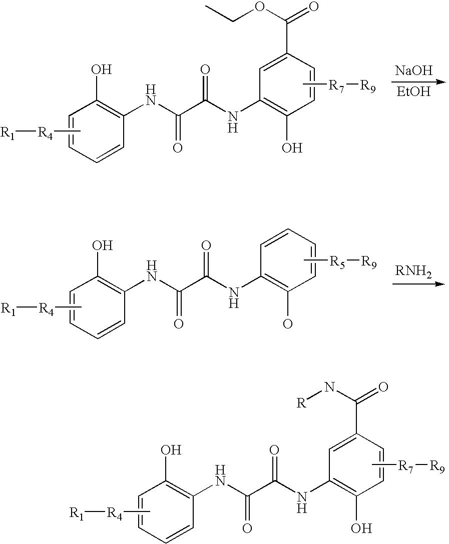

### Utility

The compounds of this invention are inhibitors of PAI-1 and are useful for the treatment or prevention of thromboembolic disorders in mammals (i.e., PAI-1 associated disorders). In general, a thromboembolic disorder is a circulatory disease caused by blood clots (i.e., diseases involving fibrin formation, platelet activation, and/or platelet aggregation). The term “thromboembolic disorders” as used herein includes arterial cardiovascular thromboembolic disorders, venous cardiovascular thromboembolic disorders, and thromboembolic disorders in the chambers of the heart. The term “thromboembolic disorders” as used herein also includes specific disorders selected from, but not limited to, unstable angina or other acute coronary syndromes, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy. The anti-thrombotic effect of compounds of the present invention is believed to be due to inhibition of PAI-1.

The effectiveness of compounds of the present invention as inhibitors of PAI-1, can be determined using a relevant purified serine protease, respectively, and an appropriate synthetic substrate. The rate of hydrolysis of the chromogenic or fluorogenic substrate by the relevant serine protease was measured both in the absence and presence of compounds of the present invention. Hydrolysis of the substrate resulted in the release of pNA (para nitroaniline), which was monitored spectrophotometrically by measuring the increase in absorbance at 405 nm. A decrease in the rate of absorbance or fluorescence change in the presence of inhibitor is indicative of enzyme inhibition. Such methods are known to one skilled in the art. The results of this assay are expressed as the inhibitory constant, Kᵢ.

Chromogenic assays for PAI-1 inhibitors were conducted using either tPA or Urokinase as a “substrate” for PAI-1 at a final assay volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. The chromogenic assay was started by adding either spectrozyme tPA substrate (American Diagnotica, Inc. CT, #444L) or S2444 from chromogenix as the substrate for urokinase. The inhibition of PAI-1 activity was determined by comparing the rate of the reactions in the absence of inhibitor, but in the presence of PAI-1. Compounds tested in the chromogenic assay are considered to be active if they exhibit Kᵢ's of equal to or less than 30 μM.

Human fibrinolysis assays were conducted by diluting the compounds of interest into assay buffer consisting of 50 mM potassium phosphate, pH 7.4 and 0.05% BSA. Human PAI-1 (Molecular Innovations Inc., Michigan, CAT #PAI-A), then tPA (Genetech, CA), human glu-plasminogen (Enzyme research laboratories (ERL), Indiana HPg2001), and plaminogen depleted human fibrinogen (ERL, HFN) were added sequentially, then followed by the addition of human thrombin (ERL, HT1002). Compounds tested in the fibrinolysis assay are considered to be active if they exhibit a Kᵢ of equal to or less than 30 μM.

Compounds of the present invention have demonstrated Kᵢ values of equal to or less than 30 μM, preferably less than 10 μM, more preferably less than 2 μM, and even more preferably less than 1.5 μM in at least one of the above assays, thereby confirming the utility of the compounds of the present invention as effective inhibitors of PAI-1 and useful for the prevention or treatment of thromboembolic disorders in mammals.

The antithrombotic effect of compounds of the present invention can be can be demonstrated using relevant in vivo thrombosis model including in a rat model of acute venous thrombosis where the thrombolytic state is induced by submaximal tPA.

In this model male SD rats (300-400 g) are anesthetized with 60 mg/kg, i.p. Na-pentobarbital. PE-205 tubing is inserted in the trachea to maintain airway patency. PE-50 catheters are inserted in both jugular veins to administer test articles and in a femoral vein to inject human recombinant tissue factor (hrTF). The vena cava is isolated by a midline abdominal incision and temperature maintained with heating lamps. A fixed stenosis is produced just distal to the renal veins by tying a ligature around a 26-gauge steel tubing that was laid alongside the vena cava segment and then removing the tubing. Thrombosis is induced with a 1.4 mL/kg infusion of hrTF (1/10 dilution RecombiPlasTin®, Ortho Diagnostics) given over 2 min. The vena cava thrombus was removed and weighed 20 min after the start of hrTF infusion.

Treatment protocol includes i.v. infusion of a PAI-1 inhibitor of the present invention followed, after 5 minutes, by an i.v. infusion of human recombinant tissue plasminogen activator (tPA, Activase, Genentech) at a submaximal dose of 10 μg/kg/min. The hrTF is administered 1 min into the tPA infusion. The PAI-1 inhibitors are administered as a loading i.v. injection plus sustaining i.v. infusion at appropriate dose levels (mg/kg+mg/kg/hr) e.g.: at 3+3 and 10+10, and 2+2 and 5+5. Both tPA and PAI-1 inhibitor infusions are maintained until thrombus removal. The administration of the compounds of the present invention to rats treated with the submaximal dose of tPA results in significantly reduced thrombus weight.

The compounds of formula I and salts thereof are inhibitors of PAI-1, a major regulatory component of the plasminogen-plasmin system. As such, compounds of the present invention are useful in the treatment, inhibition, prevention or prophylaxis in a mammal, preferably in a human, of processes involving the production and/or action of PAI-1. See e.g. Binder et al., News Physiol. Sci., 17: 56-61 (2002) and Tsikouris, J. et al., J. Clin. Pharmacol., 42:1187-1199 (2002).

Accordingly, the compounds of the present invention may also be used in treating conditions including, but not limited to, metabolic diseases correlated to triacylglycerol levels & insulin resistance such as diabetes mellitus (Type 1 & 2), hyperinsulinemia, hyperglycemia and hypertriglyciridemia; obesity, acute & chronic inflammatory lung disorder such as respiratory distress syndrome, asthma, COPD, idiopathic pulmonary fibrosis, hyperoxide lung injury and bronchopulmonary dysplasia; renal disorders such as nephritic syndrome and hemolytic uremic syndrome; and malignancies such as tumor cell invasion, metastasis and neovascularization.

The compounds of the invention are also useful for the treatment of blood and blood products used in dialysis, blood storage in the fluid phase, especially ex vivo platelet aggregation. The present compounds may also be added to human plasma during the analysis of blood chemistry in hospital settings to determine the fibrinolytic capacity thereof.

The compounds of the present invention may also be used to treat cancer including, but not limited to, breast and ovarian cancer, and as imaging agents for the identification of metastatic cancers.

The compounds of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

The compounds of the invention may be used for the treatment of myelofibrosis with myeloid metaplasia by regulating stromal cell hyperplasia and increases in extracellular matrix proteins.

The compounds of the invention may also be used in conjunction with protease inhibitor-containing highly active antiretroviral therapy (HAART) for the treatment of diseases which orginate from fibrinolytic impairment and hyper-coagulability of HIV-1 infected patients receiving such therapy.

The compounds of the invention may be used for the treatment of diabetic nephropathy and renal dialysis associated with nephropathy.

The compounds of the invention may be used to treat polycystic ovary syndrome, organ transplant rejection, septic shock and vascular damage associated with infections, cancer, septicemia, obesity, insulin resistance, proliferative diseases such as psoriasis, improving coagulation homeostasis, cerebrovascular diseases, microvascular disease, hypertension, dementia, arthritis, asthma, heart failure, arrhythmia, angina, and as a hormone replacement agent, treating, preventing or reversing progression of atherosclerosis, Alzheimer's disease, osteoporosis, osteopenia; reducing inflammatory markers, reducing C-reactive protein, or preventing or treating low grade vascular inflammation, stroke, coronary heart disease, primary and secondary prevention of myocardial infarction, stable and unstable angina, primary prevention of coronary events, secondary prevention of cardiovascular events, peripheral vascular disease, peripheral arterial disease including peripheral arterial occlusion, acute vascular syndromes, reducing the risk of undergoing a myocardial revascularization procedure, microvascular diseases such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improving coagulation homeostasis, and/or improving endothelial function, and all forms of cerebrovascular diseases.

The compounds of the invention may be used for the topical applications in wound healing for prevention of scarring.

The compounds of the present invention can be administered alone or in combination with one or more additional therapeutic agents. These include anti-coagulant or coagulation inhibitory agents, anti-platelet or platelet inhibitory agents, thrombin inhibitors, or thrombolytic or fibrinolytic agents.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e. prevent, inhibit or ameliorate) the thromboembolic disease condition or treat the progression of the disease in a host.

The compounds of the invention are preferably administered alone to a mammal in a therapeutically effective amount. However, the compounds of the invention can also be administered in combination with an additional therapeutic agent, as define below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antiviral effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

Compounds which can be administered in combination with the compounds of the present invention include, but are not limited to, anticoagulants, anti-thrombin agents, anti-platelet agents, fibrinolytics, hypolipidemic agents, antihypertensive agents, and anti-ischemic agents.

Anticoagulant agents (or coagulation inhibitory agents) that may be used in combination with the compounds of this invention include warfarin, heparin, low molecular weight heparin (for example LOVANOX™), as well as other factor Vila, VIIIa, IXa, Xa, XIa, prothrombin, TAFI, and fibrinogen inhibitors known in the art. The term anti-platelet agents (or platelet inhibitory agents), as used herein, denotes agents that inhibit platelet function such as by inhibiting the aggregation, adhesion or granular secretion of platelets. Such agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, and piroxicam, including pharmaceutically acceptable salts, hydrates or prodrugs thereof. Of the NSAIDS, aspirin (acetylsalicylic acid or ASA), and piroxicam are preferred. Other suitable anti-platelet agents include clopidrogel and ticlopidine, including pharmaceutically acceptable salts, hydrates or prodrugs thereof. Ticlopidine is also a preferred compound since it is known to be gentle on the gastro-intestinal tract in use. Still other suitable platelet inhibitory agents include IIb/IIIa antagonists, thromboxane-A2-receptor antagonists and thromboxane-A2-synthetase inhibitors, prostacyclin mimetics, phosphodiesterase (PDE) inhibitors, such as dipyridamole or cilostazol, serotonin-2-receptor antagonists, and P2Y₁ and P2Y₁₂ receptor antagonists, as well as pharmaceutically acceptable salts, hydrates or prodrugs thereof. Preferred P2Y₁₂ receptor antagonists include ticlopidine and clopidogrel, including pharmaceutically acceptable salts, hydrates or prodrugs thereof. Clopidogrel is an even more preferred agent. Ticlopidine and clopidogrel are also preferred compounds since they are known to be gentle on the gastro-intestinal tract in use.

The term thrombolytics (or fibrinolytic) agents (or thrombolytics or fibrinolytics), as used herein, denotes agents that lyse blood clots (thrombi). Such agents include tissue plasminogen activator (TPA), anistreplase, urokinase, streptokinase, PAI-1 inhibitors, and inhibitors of alpha-2-antiplasmin, including pharmaceutically acceptable salts, hydrates or prodrugs thereof. The term anistreplase, as used herein, refers to anisoylated plasminogen streptokinase activator complex, as described, for example, in European Patent Application No. 028,489, the disclosure of which is hereby incorporated herein by reference herein. The term urokinase, as used herein, is intended to denote both dual and single chain urokinase, the latter also being referred to herein as prourokinase. The term hypolipidemic agents, as used herein, includes HMG-CoA reductase inhibitors (for example, pravastatin, simvastatin, atorvastatin, and the like) and microsomal triglyceride transport protein inhibitors.

The term antihypertensive agents, as used herein, includes angiotensin-converting enzyme inhibitors (for example captopril, lisinopril, or fosinopril), angiotensin-II receptor antagonists (for example irbestatin, losartan, or valsartan), ACE/NEP inhibitors (for example omapatrilat or gemopatrilat), diuretics (for example furosemide, chlorothiazide, or amiloride) and β-blockers (for example propanolol, nadolo, or carvedilol).

Administration of the compounds of the present invention of the invention in combination with such additional therapeutic agent, may afford an efficacy advantage over the compounds and agents alone, and may do so while permitting the use of lower doses of each. A lower dosage minimizes the potential of side effects, thereby providing an increased margin of safety.

The compounds of the present invention are also useful as standard or reference compounds, for example as a quality standard or control, in tests or assays involving the inhibition of PAI-1. Such compounds may be provided in a commercial kit, for example, for use in pharmaceutical research involving PAI-1. For example, a compound of the present invention could be used as a reference in an assay to compare its known activity to a compound with an unknown activity. This would ensure the experimentor that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving PAI-1 tissue-type plasminogen activator (“tPA”) and urinary type plasminogen activator (“uPA”). For example, the presence of tPA and/or uPA in an unknown sample could be determined by addition of the relevant chromogenic substrate, for example S2444 for uPA and spectrozyme (American Diagnostics, Inc., CT #444L) for tPA, to a series of solutions containing test sample and optionally one of the compounds of the present invention. If production of pNA is observed in the solutions containing test sample together with exogenous PAI-1 and a compound of the present invention, then one would conclude that selective PAI-1 activity was absent, rather than, for example, PAI-2 or PAI-3 activity.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt or hydrate form thereof; and (c) a package insert stating that the pharmaceutical composition can be used for the treatment of a thromboembolic disorder (as defined previously). In another embodiment, the package insert states that the pharmaceutical composition can be used in combination (as defined previously) with a second therapeutic agent to treat a thromboembolic disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Biological Assays

Chromogenic Assays for Inhibitors of PAI-1.

Chromogenic assays for PAI-1 inhibitors were conducted in 96 well plates (Costar 25381-054). Reactions were conducted using either tPA or Urokinase as a “substrate” for PAI-1. Reactions were set up such that each well contained a final volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. Each well contained 50 uL of buffer (50 mM Tris pH 8.3, 0.1 M NaCl containing 100 uL of Tween 80/L; buffer was filtered using a 0.2 um filter) and 2 uL of each compound of interest which had been diluted in 100% DMSO. Next, 50 uL of 10.4 nM PAI-1, in buffer, was added and the plate was vortexed for 1 min followed by a 30 minute incubation at room temperature. Fifty microliters of either 8 nM t-PA or 40 units/ml urokinase was added and the plate was vortexed for 1 minute. The chromogenic assay was started by adding either 50 uL of 1 mM spectrozyme tPA substrate (American Diagnotica, Inc. CT, #444L) or 50 uL of the 0.4 mM S2444 from chromogenix as the substrate for urokinase. The absorbance was measured at 405 nm over 15 mins using the kinetic mode of a spectramax 190 plate reader (Molecular Devices). The inhibition of PAI-1 activity was determined by comparing the rate of the reactions in the absence of inhibitor, but in the presence of PAI-1.

Human Fibrinolysis Assay Protocol

Human fibrinolysis assays were conducted in 96 well plates (Costar 25381-054). Stock solutions were diluted into assay buffer consisting of 50 mM potassium phosphate, pH 7.4 and 0.05% BSA. Compounds to be tested were serially diluted in 100% DMSO. Each well contained 50 uL assay buffer and 2 uL of a given concentration of the compound of interest. Fifty microliters of a 2 nM human PAI-1 (Molecular Innovations Inc., Michigan, CAT #PAI-A) was added to each well and the plate was incubated at room temperature for 5 minutes. Next, 50 uL of each of the following solutions was added to each well and mixed for one minute: 0.42 nM tPA (Genetech, CA), 800 nM human glu-plasminogen (Enzyme research laboratories (ERL), Indiana HPg2001), 2 mg/ml plaminogen depleted human fibrinogen (ERL, HFN). Finally, the reaction was initiated by the addition of 50 uL of 14 nM human thrombin (ERL, HT1002). The time course of the reaction was followed by measuring the absorbance at 405 nm using a spectramax plate reader in kinetic mode. The time course of the reaction was observed for 4 hours. The extended time course was used to monitor the shape of the reaction curve. The percent inhibition of the fibrinolysis was determined by comparing the absorbance values at the 4 hour time point for wells containing compounds and control wells which contained no compound.

### EXAMPLES

The following Examples illustrate embodiments of the inventive compounds and starting materials, and are not intended to limit the scope of the claims. For ease of reference, the following abbreviations are used herein:

### Abbreviations

CH₃CN=acetonitrile
DCC=dicyclohexylcarbodiimide
DCE=dichloroethane
DCM=dichloromethane
DMAP=4-dimethylaminopyridine
DIPEA or DIEA=N,N-diisopropylethylamine
DME=1,2-dimethoxyethane
DMF=dimethyl formamide
EDCI=1-3-dimethylaminopropyl)-3-ethylcarbodiimide
Et₂O=diethyl ether
HOBT=1-hydroxybenzotriazole
EtOAc=ethyl acetate
HCl=hydrochloric acid
KOH=potassium hydroxide
K₂CO₃=potassium carbonate
LiAlH₄=lithium aluminum hydride
MeCN=acetonitrile
MeOH=methanol
MgSO₄=magnesium sulfate
NaH=sodium hydride
NaOH=sodium hydroxide
NMP=1-methyl-2-pyrrolidinone
SOCl₂=thionyl chloride
TEA=triethylamine
THF=tetrahydrofurane
bp=boiling point
g=gram(s)
mg=milligram(s)
ml=milliliter
μl=microliter
l=liter
mmol=millimole
μmol=micromole
mol=mole
mp=melting point
RT=room temperature
NMR (Nuclear Magnetic resonnance was performed on a Brucker 200 spectrometer (s=singulet, d=doublet, t=triplet, dd=doublet of doublet, m=multiplet) Elementary analysis were carried on a Carlo-Erba Mod 106 elementary analyzer

### Example 1

### N,N′-Bis-(3,5-dichloro-2-hydroxy-phenyl)-oxalamide

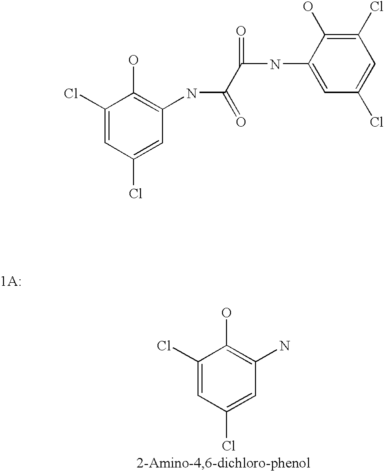

60 g of 4,6 dichloro-2 nitrophenol were dissolved in 600 ml methanol and 5 g. Raney nickel were added. This solution was hydrogenated under atmospheric pressure for 4 hours and filtrated. After evaporation of methanol, the residue was crystallized in isopropyl ether to give 40.5 g of IA as a grey solid (mp=33° C.).

Title Compound:

To 1 g of a solution of 1A in 20 ml THF was added 0.25 ml of oxalyldichloride. The mixture was stirred at RT for 1 h, filtrated and the precipitate was washed with acetone to give 330 mg of Example 1 as a white product, melting at 325° C.

Microanalysis: theory (%): C, 41.0; H, 1.97; N, 6.83. obtained (%): C, 41.04; H, 2.00; N, 6.63.

### Example 2

### N-(3,5-Dichloro-2-hydroxy-phenyl)-N′-(2-hydroxy-phenyl)-oxalamide

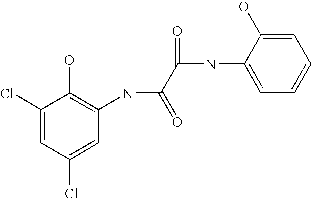

### 2A: N-(3,5-Dichloro-2-hydroxy-phenyl)-oxalamic Acid Ethyl Ester

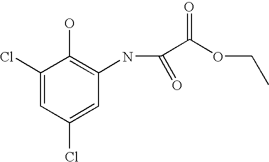

25 g of 1A was dissolved in 250 ml THF and the solution was cooled at 5° C. 2-chloroethylchloroformate (16 ml) were added dropwise under cooling and then the reaction mixture was allowed to return to RT and stirred for 8 h. The solution mixture was the concentrated to half-volume and isopropyl ether was added. The precipitate was filtrated out and washed with isopropyl ether to give 24 g of a white-off solid. (mp: 190° C.). ¹H NMR (DMSO-d6): 10.9 (1H,s)), 7.82 (1H, d, J=2 Hz), 7.38 (1H, d, J=2 Hz), 4.30 (2H, d, J=9 Hz, 1.35 (3H, t, J=9 Hz).

Title Compound

To 1 g of 2A in dioxane was added 400 mg of 2 aminophenol. The mixture was refluxed in dioxane for 6 h. and then left to return at RT. The obtained precipitate was collected and washed with acetone, then methanol to give 400 mg of Example 2 as crystals which melted at 298° C. Microanalysis: theory (%): C, 49.3; H, 2.95; N, 8.21. obtained (%): C, 49.0; H, 2.97; N, 8.09.

### Examples 3 to 15

Examples 3 to 15 were prepared according to the procedures described in Example 2 using the appropriate starting materials (commercially available anilines).

### Example 3

### N-(3,5-Dichloro-2-hydroxy-phenyl)-N′-(2-hydroxy-4-methyl-phenyl)-oxalamide

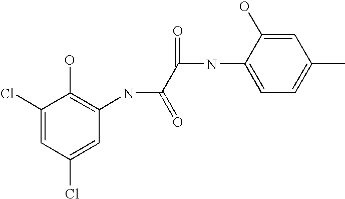

Mp=283° C.;

Microanalysis: theory (%): C, 50.7; H, 3.41; N, 7.89. obtained (%): C, 50.5; H, 3.36; N, 7.68.

### Example 4

### N-(3,5-Dichloro-2-hydroxy-phenyl)-N′-(2-hydroxy-5-nitro-phenyl)-oxalamide

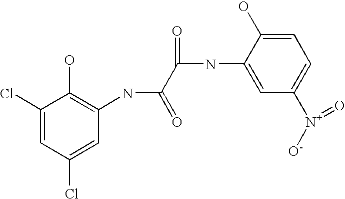

Mp=386° C.

Microanalysis: theory (%): C, 43.5; H, 2.35; N: 10.9. obtained (%): C, 43.3; H, 2.48; N, 10.6.

### Example 5

### N-(3,5-Dichloro-2-hydroxy-phenyl)-N′-(4-hydroxy-biphenyl-3-yl)-oxalamide

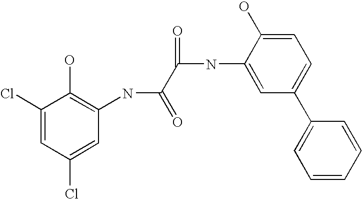

Mp: 288° C.

Microanalysis: theory (%): C, 57.5; H, 3.38; N, 6.7. obtained (%): C, 57.3; H, 3.1; N, 6.6.

### Example 6

### N-(3,5-Dichloro-2-hydroxy-phenyl)-N′-(3-hydroxy-naphthalen-2-yl)-oxalamide

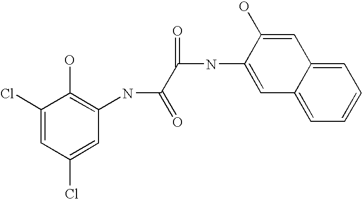

Mp: 313° C.

Microanalysis: theory (%): C, 55.3; H, 3.09; N, 7.16. obtained (%): C, 54.7; H, 2.76; N, 7.03.

### Example 7

### N-(5-Chloro-2-hydroxy-phenyl)-N′-(3,5-dichloro-2-hydroxy-phenyl)-oxalamide

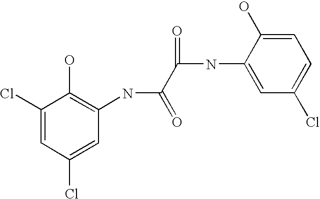

Mp: 311° C.

Microanalysis: theory (%): C, 44.8; H, 2.41; N, 7.46. obtained (%): C, 44.7; H, 2.41; N, 7.15.

### Example 8

### N-(3,5-Dichloro-2-hydroxy-phenyl)-N′-(2-hydroxy-naphthalen-1-yl)-oxalamide

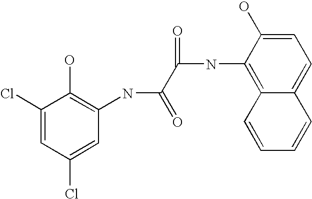

Mp: 265° C.

Microanalysis: theory (%): C, 55.3; H, 3.09; N, 7.16. obtained (%): C, 54.8; H, 3.16; N, 6.90.

### Example 9

### N-(3,5-Dichloro-2-hydroxy-phenyl)-N′-[4-(1,1-dimethyl-propyl)-2-hydroxy-phenyl]-oxalamide

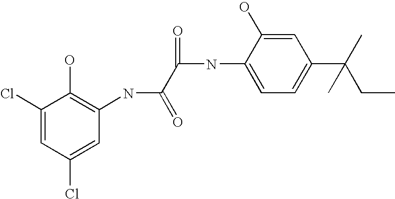

Mp: 260° C.

Microanalysis: theory (%): C, 55.5; H, 4.90; N, 6.81. obtained (%): C, 55.4; H, 4.68; N, 6.76.

### Example 10

### N-(3,5-Dichloro-2-hydroxy-phenyl)-N′-(2-hydroxy-6-methyl-phenyl)-oxalamide

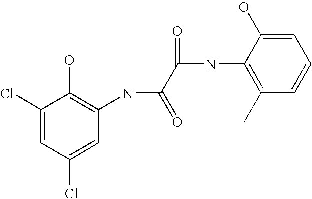

Mp: 260° C.

Microanalysis: theory (%): C, 50.7; H, 3.81; N, 7.88. obtained (%): C, 50.4; H, 3.53; N, 7.60.

### Example 11

### N-(5-Chloro-2-hydroxy-4-nitro-phenyl)-N′-(3,5-dichloro-2-hydroxy-phenyl)-oxalamide

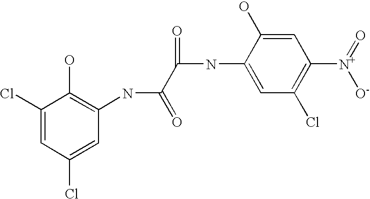

Mp: 289

Microanalysis: theory (%): C, 40.0; H, 1.92; N, 9.99. obtained (%): C, 39.7; H, 1.97; N, 9.56.

### Example 12

### 4-[(3,5-Dichloro-2-hydroxy-phenylaminooxalyl)-amino]-3-hydroxy-benzoic Acid Methyl Ester

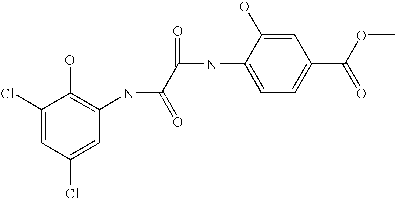

Mp: 271° C.

Microanalysis: theory (%): C, 48.1; H, 3.03; N, 7.02. obtained (%): C, 47.7; H, 3.08; N, 6.68.

### Example 13

### N-(3,5-Dichloro-2-hydroxy-4-methyl-phenyl)-N′-(3,5-dichloro-2-hydroxy-phenyl)-oxalamide

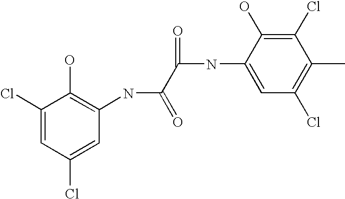

Mp: 305

Microanalysis: theory (%): C, 42.4; H, 2.38; N, 6.61. obtained (%): C, 42.4; H, 2.46; N, 6.46.

### Example 14

### N-(3,5-Dichloro-2-hydroxy-phenyl)-N′-(5-ethanesulfonyl-2-hydroxy-phenyl)-oxalamide

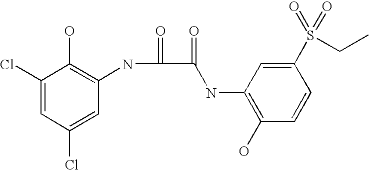

Mp: 272

Microanalysis: theory (%): C, 44.3; H, 3.26; N, 6.47. obtained (%): C, 43.9; H, 3.14; N, 6.31.

### Example 15

### N-(5-Cyano-2-hydroxy-phenyl)-N′-(3,5-dichloro-2-hydroxy-phenyl)-oxalamide

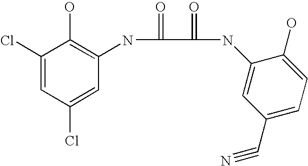

Mp: 298° C.

Microanalysis: theory (%): C, 49.2; H, 2.48; N, 11.5. obtained (%): C, 48.8; H, 2.32; N, 11.1.

### Example 16

### N-(3,5-Dichloro-2-hydroxy-phenyl)-N′-(2-hydroxy-4-nitro-phenyl)-oxalamide

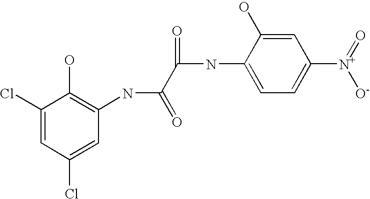

2A (0.7 g) was dissolved in 25 ml xylene, 0.39 g of 2-amino-5 nitrophenol were added and the mixture was refluxed for 6 hours. The obtained precipitate was filtrated and washed by acetone to give 120 mg of Example 16 melting at 290° C. Microanalysis: theory (%): C, 43.5; H, 2.35; N, 10.9. obtained (%): C, 43.2; H, 2.40; N, 10.7.

### Examples 17 to 21

Examples 17 to 21 were obtained according to the procedure of example 16 via 2A and appropriate starting materials (commercially available anilines).

### Example 17

### N-(3,5-Dichloro-2-hydroxy-phenyl)-N′-(2-hydroxy-5-methoxy-phenyl)-oxalamide

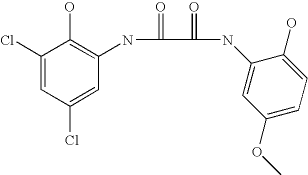

Mp: 273° C.

Microanalysis: theory (%): C, 48.5; H, 3.28; N, 7.55. obtained (%): C, 48.1; H, 3.26; N, 7.45.

### Example 18

### N-(3,5-Dichloro-2-hydroxy-phenyl)-N′-(5-fluoro-2-hydroxy-phenyl)-oxalamide

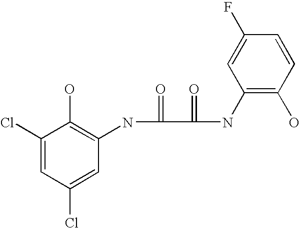

Mp: 297° C.

Microanalysis: theory (%): C, 46.8; H, 2.52; N, 7.80. obtained (%): C, 46.5; H, 2.73; N, 7.41.

### Example 19

### N-(3-Chloro-2-hydroxy-phenyl)-N′-(3,5-dichloro-2-hydroxy-phenyl)-oxalamide

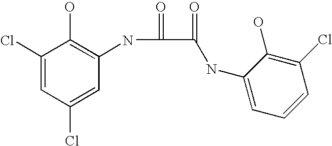

Mp: 296° C.

Microanalysis: theory (%): C, 44.8; H, 2.41; N, 7.46. obtained (%): C, 44.9; H, 2.49; N, 7.24.

### Example 20

### N-(3,5-Dichloro-2-hydroxy-phenyl)-N′-(3,4-difluoro-2-hydroxy-phenyl)-oxalamide

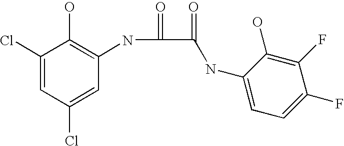

Mp: 302° C.

Microanalysis: theory (%): C, 44.6; H, 2.24; N, 7.43. obtained (%): C, 44.3; H, 2.34; N, 7.28.

### Example 21

### N-(3,5-Dichloro-2-hydroxy-phenyl)-N′-(2-hydroxy-5-trifluoromethyl-phenyl)-oxalamide

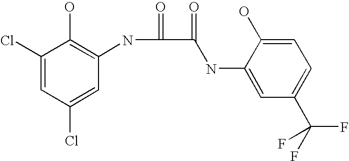

Mp: 281° C.

Microanalysis: theory (%): C, 44.0; H, 2.22; N: 6.85. obtained (%): C, 43.7; H, 2.27; N, 6.50.

### Example 22

### N-(3,5-Dichloro-2-hydroxy-phenyl)-N′-(2-hydroxy-5-sulfamoyl-phenyl)-oxalamide

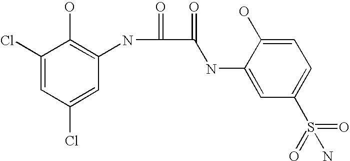

### 22A: 6,8-Dichloro-4H-benzo[1,4]oxazine-2,3-dione

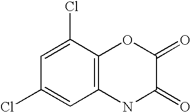

25.3 g of 1A was dissolved in 250 ml dichloromethane and 40 ml triethylamine were added. The mixture was cooled by an ice-water bath and then 12.7 ml of oxalyldichloride were added. Temperature was left to return to RT and the mixture was stirred 3 h. at RT. 100 ml water were added and the obtained precipitate was filtrated out and washed by dichloromethane to yield 17 g of raw product which were recristallized in 500 ml acetonitrile to give 9.5 g of 22A as an off-white product (mp: 303° C.) ¹H NMR (DMSO-d6): 12 (s,1H)), 7.33 (1H, d, J=2 Hz), 7.05 (1H, t, J=2 Hz)

Title Compound

500 mg of the product of 22A and 400 mg of 2-amino-5-sulfonamido phenol were dissolved in 100 ml THF and refluxed for 4 hours. The obtained crystals were washed with acetone to obtain 225 mg of a product melting at 312° C. Microanalysis theory (%): C, 40.0; H, 2.64; N, 10.0. obtained (%): C, 40.0; H, 2.60; N, 9.85.

### Example 23 to 25

The following products were obtained from the product of 22A and appropriate starting materials (commercially available anilines) by the procedure of Example 22.

### Example 23

### 3-[(3,5-Dichloro-2-hydroxy-phenylaminooxalyl)-amino]-4-hydroxy-benzoic Acid

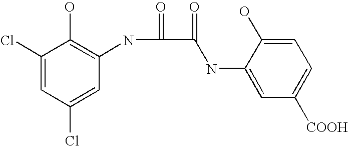

Mp: 340

Microanalysis: theory (%): C, 46.8; H, 2.62; N 7.27. obtained (%): C, 46.6; H, 2.73; N, 7.27.

### Example 24

### 4-[(3,5-Dichloro-2-hydroxy-phenylaminooxalyl)-amino]-3-hydroxy-benzoic Acid

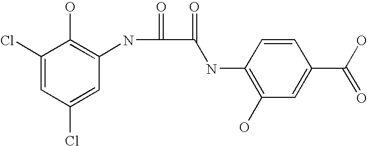

Mp: 323° C.

Microanalysis: theory (%): C, 46.8; H, 2.62; N, 7.27. obtained (%): C, 46.8; H, 2.74; N, 7.11.

### Example 25

### 3-[(3,5-Dichloro-2-hydroxy-phenylaminooxalyl)-amino]-4-hydroxy-benzoic Acid Ethyl Ester

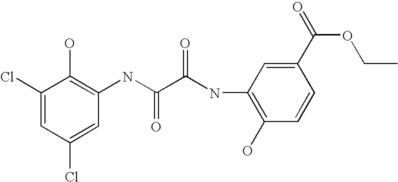

Mp: 270° C.

Microanalysis: theory (%): C, 49.4; H, 3.41; N, 6.78. obtained (%): C, 49.0; H, 3.65; N, 6.79.

### Example 26

### N-(3,5-Dichloro-2-hydroxy-phenyl)-N′-(3-fluoro-2-hydroxy-phenyl)-oxalamide

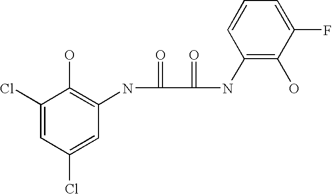

### 26A: 2-Amino-6-fluoro-phenol

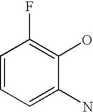

6 nitro-2-fluorophenol (4.6 g) were dissolved in methanol, Raney nickel was added and the mixture was submitted to hydrogenation at atmospheric pressure until the absorption was complete. The mixture was filtrated, the filtrate was filtrated on activated coal and evaporated to give a residue which was crystallized in ether to yield 2 g of 26A which melted at 115° C. and used without further purification in the next step.

### 26B: N-(3-Fluoro-2-hydroxy-phenyl)-oxalamic Acid Ethyl Ester

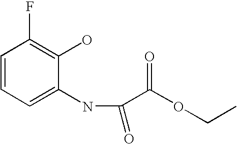

26A (2 g) was dissolved in 30 ml THF, and the mixture was cooled by an ice-water bath. 2 ml of 2-chloroacetylchoride were dropped and the mixture was stirred for 3 hours and left to return at RT. The mixture was concentrated to half of its initial volume, ether was added, and after filtration were obtained 2.4 g of 26B as crystals which melted at 168° C. and were used in the next step without further purification.

Title Compound

26B (1 g) and 0.9 g of the product of 2A were dissolved in xylene and the mixture was refluxed for 18 hours. The hot mixture was filtrated and the precipitate washed with acetone to give 400 mg of a product melting at 298° C.

Microanalysis: theory (%): C, 46.8; H, 2.52; N: 7.80. obtained (%): C, 46.6; H, 2.64; N, 7.73.

### Example 27

### N-(3,5-Dichloro-2-hydroxy-phenyl)-N′-(2,3,5-trichloro-6-hydroxy-phenyl)-oxalamide

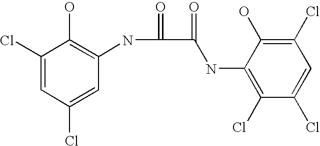

### 27A: N-(3,4,6-Trichloro-2-hydroxy-phenyl)-oxalamic Acid Ethyl Ester

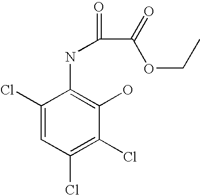

2 g of 2 amino-3,4,6 trichlorophenol were dissolved in 20 ml THF, the mixture was cooled by an ice-water bath and 1.1 ml of 2-chloroacetylchoride were dropped. The mixture was stirred for 3 hours and left returning to RT. The solvent was evaporated and the residue taken in isopropyl ether and in acetone to yield 310 mg of 27A which melted at 178° C. and was used in the next step without purification.

Title Compound

310 mg of the product of 27A and 200 mg of 2A were dissolved in xylene and refluxed for 10 hours. The mixture was left to return to RT, filtrated, and the collected crystals were washed with dichloromethane to yield crystals melting at 280° C.

Microanalysis: theory (%): C, 37.8; H, 1.59; N, 6.30. obtained (%): C, 38.8; H, 1.59; N, 6.21.

### Example 28

### N,N′-Bis-(3,5-dichloro-2-hydroxy-4-methyl-phenyl)-oxalamide

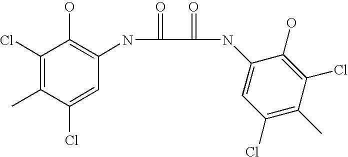

2.6 g of 6 amino-2,4-dichlorophenol were dissolved in 40 ml THF and 0.45 ml of oxalyldichloride were dropped at RT. The mixture was stirred for 3 hours and the obtained precipitate was filtrated, washed with water and then acetone to yield crystals melting at 330° C.

Microanalysis: theory (%): C, 43.9; H, 2.76; N, 6.40. obtained (%): C, 44.1; H, 2.80; N, 6.28.

### Example 29

### N-(3,5-Dichloro-2-hydroxy-phenyl)-N′-(2-hydroxy-5-phenethylcarbamoyl-phenyl)-oxalamide

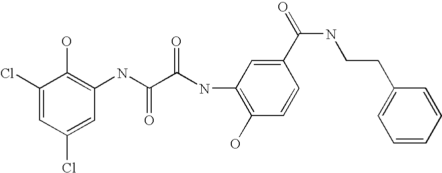

960 mg of the product of Example 23 were dissolved in DMF and 380 mg of HOBT and 0.55 ml of DIEA were added. The mixture was stirred at RT for 30 nm and then 700 mg of phenethylamine were added. The reaction mixture was stirred for 2 hours at RT then 1 h at 50° c. After returning to RT, diluted HCl was added, the precipitate filtered, washed with a solution 1N NH₄OH, then with acetone and methanol to obtain 45 mg of a product melting at 265° C.

Microanalysis: theory (%): C, 56.6; H, 3.92; N, 8.60. obtained (%): C, 56.3; H, 4.15; N, 8.42.

The products of Example 30 to Example 36 were obtained starting from the product of Example 23 and the appropriate starting materials (commercially available amines) by the procedure described for Example 29:

### Example 30

### N-(3,5-Dichloro-2-hydroxy-phenyl)-N′-{2-hydroxy-5-[3-(4-methyl-piperazin-1-yl)-propylcarbamoyl]-phenyl}-oxalamide

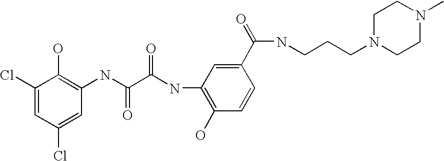

Mp: 232° C.

Microanalysis: theory (%, 2H₂O): C, 49.3; H, 5.3; N, 12.3. obtained (%): C, 48.9; H, 5.53; N, 12.5.

### Example 31

### {3-[(3,5-Dichloro-2-hydroxy-phenylaminooxalyl)-amino]-4-hydroxy-benzoylamino}-acetic Acid Ethyl Ester

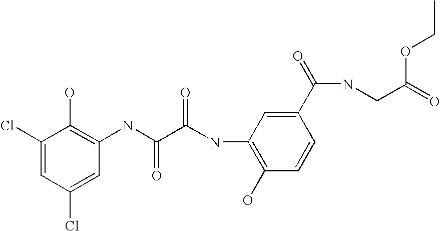

Mp: 242

¹H NMR (DMSO-d6): 10.26 (s,1H), 9.98 (s,1H), 8.86 (t,1H), 8.56 (s, 1H), 8.0 (d, 1H), 7.65 (d, 1H), 7.40 (d,1H), 7.04 (d, 1H), 4.21 (qdt, 2H), 3.98 (t, 2H) 1.10 (t, 3H)

### Example 32

### 2(2R)-{3-[(3,5-Dichloro-2-hydroxy-phenylaminooxalyl)-amino]-4-hydroxy-benzoylamino}-3-phenyl-propionic acid ethyl ester

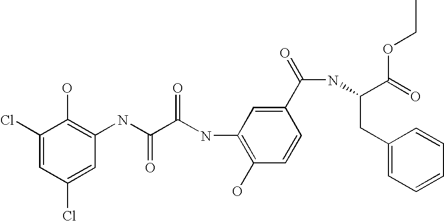

Mp: 254° C.

Microanalysis: theory (%, 0.5H₂O): C, 54.8; H, 4.25; N, 7.39. obtained (%): C, 55.0; H, 4.39; N, 7.70.

### Example 33

### N-(3,5-Dichloro-2-hydroxy-phenyl)-N′-{2-hydroxy-5-[(pyridin-4-ylmethyl)-carbamoyl]-phenyl}-oxalamide

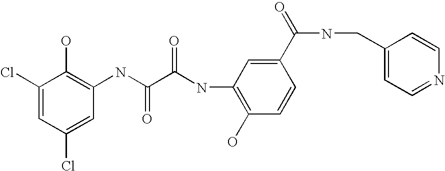

Mp: 250° C.

¹H NMR (DMSO-d6): 9.85 (t, 1H), 8.67 (s, 1H), 8.5 (d, 2H), 8.0 (s, 1H), 7.70 (dd, 1H), 7.31 (d, 1H), 7.32 (d, 1H), 7.00 (d, 1H), 4.48 (d, 2H)

### Example 34

### N-(3,5-Dichloro-2-hydroxy-phenyl)-N′-{2-hydroxy-5-[2-(1-methyl-pyrrolidin-2-yl)-ethylcarbamoyl]-phenyl}-oxalamide (Racemic)

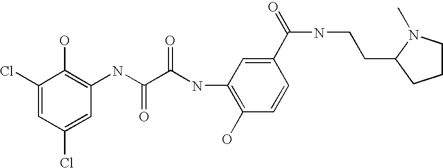

Mp: 220° C.

Microanalysis: theory (%): C, 53.3; H, 4.88; N, 9.59. obtained (%): C, 53.0; H, 4.57; N, 9.35.

### Example 35

### N-(3,5-Dichloro-2-hydroxy-phenyl)-N′-[5-(3-dimethylamino-propylcarbamoyl)-2-hydroxy-phenyl]-oxalamide

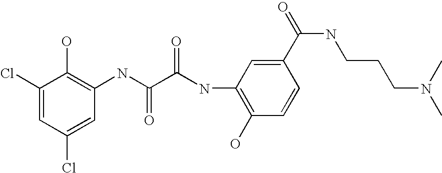

Mp: 205° C.

Microanalysis: theory (%, 1H₂O): C, 49.3; H, 4.96; N, 11.5. obtained (%): C, 48.9; H, 4.69; N, 11.2.

### Example 36

### N-(3,5-Dichloro-2-hydroxy-phenyl)-N′-[2-hydroxy-5-(3-imidazol-1-yl-propylcarbamoyl)-phenyl]-oxalamide

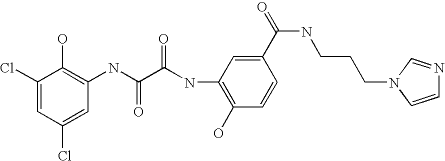

Mp: 175° C.

Microanalysis: theory (%, 1.5H₂O): C, 48.6; H, 4.27; N, 13.5. obtained (%): C, 48.4; H, 4.0; N, 13.9.

### Example 37

### Hydrochloride of N-(3,5-Dichloro-2-hydroxy-phenyl)-N′-[5-(2-dimethylamino-ethylcarbamoyl)-2-hydroxy-phenyl]-oxalamide

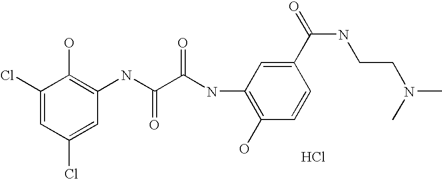

1.5 g of the product of Example 23 were dissolved in THF and 600 mg of HOBT and 0.8 ml of DIEA were added. The mixture was stirred at RT for 30 nm and then 700 mg (2 equivalent) of N,N dimethylethylamine were added. The reaction mixture was stirred for 2 hours at RT then 1 h at 50° c. After returning to RT, THF was evaporated and the residue taken in diluted HCl, filtered, washed with acetone and the collected rystals were recrystallized in DMF to yield 110 mg of a product melting over 350° C.

Microanalysis: theory (%, 0.5H₂O)): C, 45.6; H, 4.43; N, 11.2. obtained (%): C, 45.5; H, 4.66; N, 11.5.

### Example 38

### Hydrochloride of N-(3,5-Dichloro-2-hydroxy-phenyl)-N′-{2-hydroxy-5-[(pyridin-2-ylmethyl)-carbamoyl]-phenyl}-oxalamide

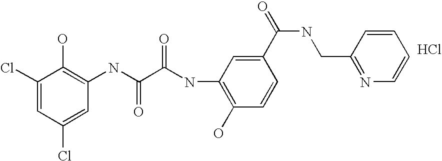

The product of Example 38 was obtained from the product of Example 23 using the procedure of Example 37.

Mp>350

Microanalysis: theory (%, 1H₂O): C, 47.9; H, 3.61; N, 10.6. obtained (%): C, 47.9; H, 3.57; N, 10.5.

### Example 39

### {3-[(3,5-Dichloro-2-hydroxy-phenylaminooxalyl)-amino]-4-hydroxy-benzoylamino}-acetic Acid

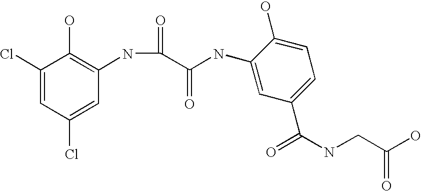

The product of Example 31 (200 mg) was dissolved in EtOH and 4 ml of NaOH (1N) was added. The mixture was heated to 50° C. and stirred for 40 nm. A precipitate appeared, wich was filtered, dissolved in water and concentrated HCl was added. The obtained precipitate was filtered and dried to give 50 mg of a product melting at 273° C.

Microanalysis: theory (%, 1H₂O): C, 52.4; H, 3.85; N: 7.64. obtained (%): C, 52.4; H, 3.72; N, 7.78.

### Example 40

### 2(2R)-{3-[(3,5-Dichloro-2-hydroxy-phenylaminooxalyl)-amino]-4-hydroxy-benzoylamino}-3-phenyl-propionic Acid

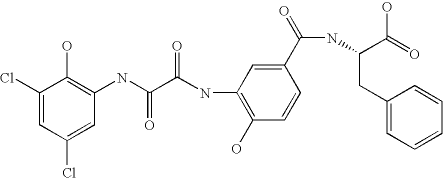

Example 40 was obtained starting from Example 32 using the same procedure than for Example 39.

Mp: 251° C.

Microanalysis: theory (%, 1H₂O): C, 52.4; H, 3.85; N, 7.64. obtained (%): C, 52.4; H, 3.70; N, 7.78.

### Example 41

### N-(3,5-Dichloro-2-hydroxy-phenyl)-N′-[2-hydroxy-4-(3-morpholin-4-yl-propylcarbamoyl)-phenyl]-oxalamide

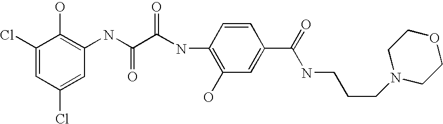

500 mg of the product of Example 24 were dissolved in DMF and 250 mg HOBt, 300 ml DIEA and 380 mg (2 equivalents) of 1-aminopropylmorpholine were added. The mixture was stirred for one hour at RT and then for 2 hours at 50° C. After return to RT, water was added and the obtained precipitate was collected, washed with water and acetone to give 50 mg of a product melting at 227° C.

Microanalysis: theory (%, 1H₂O): C, 49.1; H, 5.05; N, 10.5. obtained (%): C, 48.9; H, 5.04; N, 10.4.

Examples 42 and 43 were obtained from the product of Example 24 and commercially available amines according to the procedure of Example 41

### Example 42

### N-(3,5-Dichloro-2-hydroxy-phenyl)-N′-[4-(3-dimethylamino-propylcarbamoyl)-2-hydroxy-phenyl]-oxalamide

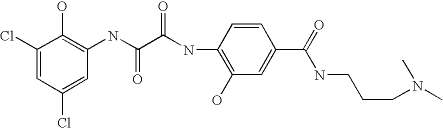

Mp: 205

Microanalysis: theory (%, 2H₂O): C, 47.5; H, 5.18; N, 11.1. obtained (%): C, 47.7; H, 4.96; N, 11.1.

### Example 43

### N-(3,5-Dichloro-2-hydroxy-phenyl)-N′-[2-hydroxy-4-(3-imidazol-1-yl-propylcarbamoyl)-phenyl]-oxalamide

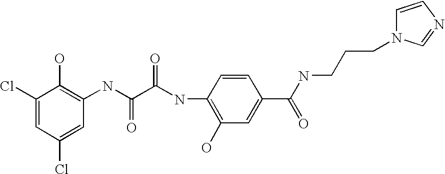

Mp: 225° C.

¹H NMR (DMSO-d6): 10.0 (s,1H), 8.50 (s,1H), 8.27′d, 1H), 8.12(d, 1H), 7.99 (D, 1H), 7.75 (s,1H), 7.49 (s, 1H), 7.39(d, 1H), 7.38(s, 1H), 7.13(d, 1H), 7.01(s, 1H), 4.15(t, 2H), 3.25 (t, 2H), 1.95 (m, 2H)

### Example 44

### N-(5-Amino-2-hydroxy-phenyl)-N′-(3,5-dichloro-2-hydroxy-phenyl)-oxalamide

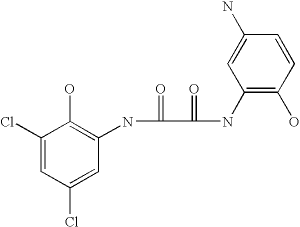

150 mg of the product of Example 4 were dissolved in THF, Raney nickel was added and the mixture hydrogenated at RT for 3 hours. THF was evaporated and the residue was crystallized in ethanol to give 45 mg of a product melting over 350° C.

Microanalysis: theory (%): C, 47.2; H, 3.11; N, 11.8. obtained (%): C, 46.9; H, 3.21; N, 11.8.

### Example 45

### N,N′-Bis-(5-chloro-2-hydroxy-4-nitro-phenyl)-oxalamide

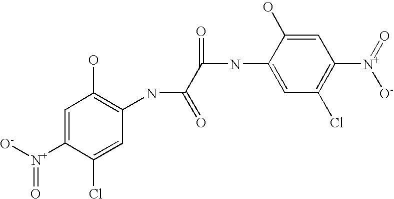

19.9 g of 2 amino-4-chloro-5-nitrophenol were dissolved in 150 ml of THF and the mixture was cooled by a ice-water bath. 8.7 ml of oxalyl chloride were added dropwise and the mixture was stirred at RT for 8 hours. Water was then added and a precipitate was obtained which was washed with acetone to give 14.5 g of a product melting at 302° C.

Microanalysis: theory (%): C, 39.0; H, 1.87; N, 13.0. obtained (%): C, 39.1; H, 1.81; N, 13.1.

### Example 46

### N,N′-Bis-(4-amino-5-chloro-2-hydroxy-phenyl)-oxalamide dihydrochloride

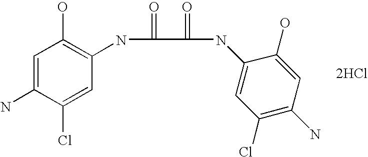

4.3 g of the product of Example 45 were dissolved in methoxyethanol and hydrogenated over Raney nickel. After completion of the absorption, methoxyethanol was evaporated, and the residue crystallized in dichloromethane. The obtained crystals were dissolved in DMF and isopropyl alcohol containing HCl was added. The obtained crystals were washed with acetone to give 240 mg of a product melting at 360° C.

Microanalysis: theory (%, 2DMF): C, 40.7; H, 4.78; N, 14.2. obtained (%): C, 40.5; H, 4.6; N, 14.0.

### Example 47

### 2-[(3,5-Dichloro-2-hydroxy-phenylaminooxalyl)-amino]-4-methyl-thiophene-3-carboxylic Acid Ethyl Ester

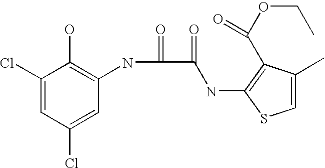

To a solution of 1 g of ethyl-2-amino-4-methyl thiophen3-carboxylic acid in dichloromethane was added dropwise 5 ml of oxalyl chloride. The reaction was stirred one night at RT, then the dichloromethane was evaporated, the residue taken in THF and one equivalent of a solution of the product of Preparation 1 was added. After stirring for five hours at RT, THF was evaporated and the residue crystallized in acetone to give 800 mg of a off-white product melting at 242° C.

Microanalysis: theory (%): C, 46.0; H, 3.38; N, 6.71. obtained (%): C, 45.6, H, 3.34; N, 6.53.

### Example 48

### N-(3,5-Dichloro-2-hydroxy-phenyl)-N′-(3,5-dichloro-pyridin-2-yl)-oxalamide

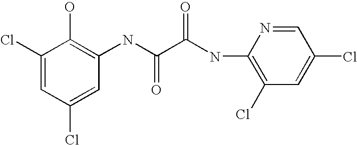

To a solution of 1 g of 2-amino-3,5 dichloropyridine in dichloromethane was added dropwise 5 ml of oxalyl chloride. The reaction was stirred one night at RT, then the dichloromethane was evaporated, the residue taken in THF and one equivalent of a solution of the product of Preparation 1 was added. After stirring for five hours at RT, THF was evaporated and the residue crystallized in acetone to give 800 mg of a off-white product melting at 245° C.

Microanalysis: theory (%): C, 39.5; H, 1.18; N, 10.6. obtained (%): C, 39.7; H, 1.34; N, 10.65.

1. A method of treating a disorder associated with high levels of PAI-1 comprising administering to a patient in need thereof a therapeutically effective amount of at least one compound of formula (I), 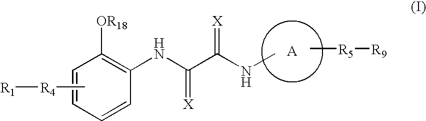
or a pharmaceutically-acceptable salt, prodrug, stereoismer or solvate thereof, wherein:
A is aryl or heteroaryl;
X is O or S;
R₁-R₉ are independently selected from hydrogen, alkyl, substituted alkyl, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀, —N₁₃C(═O)NR₁₁R₁₂, halogen, nitro and cyano;
or any two of R₁-R₉ located on neighboring carbon atoms of the phenyl ring may be taken together to form a fused ring system in combination with the phenyl ring to which they are attached, wherein the fused ring system may be optionally further substituted;
R₁₀, R₁₁, R₁₂ and R₁₃ are independently selected from hydrogen, alkyl, substituted alkyl, aryl, heteroaryl, cycloalkyl and heterocyclo, wherein each instance of R₁₀, R₁₁, R₁₂ and/or R₁₃ is selected independently; and
R₁₈ is hydrogen, alkyl or substituted alkyl.

2. The method according to claim 2, comprising administering to a patient in need thereof a therapeutically effective amount of at least one compound having the formula (Ia), 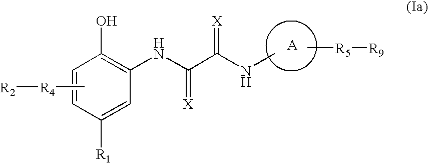
or a pharmaceutically-acceptable salt, prodrug, stereoisomer or solvate thereof, wherein:
X is O or S;
R₁ is halogen; and
R₂-R₄ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
R₅ is —OR₁₇ or —SR₁₇;
R₆-R₉ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
or any two of R₆-R₉ located on neighboring carbon atoms of the phenyl ring may be taken together to form a fused ring system in combination with the phenyl ring, wherein the fused ring system may be optionally further substituted; and
R₁₇ is hydrogen, alkyl, substituted alkyl, cycloalkyl, aryl heteroaryl or heterocyclo.

3. A method according to claim 2, comprising administering to a patient in need thereof a therapeutically effective amount of at least one compound, or a pharmaceutically-acceptable salt, prodrug, stereoismer or solvate thereof, in which:
R₁₇ is hydrogen or C₁₋₆alkyl.

4. A method according to claim 2 comprising administering to a patient in need thereof a therapeutically effective amount of at least one compound, or a pharmaceutically-acceptable salt, prodrug, stereoismer or solvate thereof, in which R₂—R₄ and R₆-R₉ are independently selected from hydrogen, alkyl, substituted alkyl, halogen, nitro, cyano, —OR₁₀, —OC(═O)R₁₀, —CO₂R₁₀, —C(═O)NR₁₁R₁₂, —NR₁₁R₁₂, —S(═O)R₁₀, —SO₂R₁₀, and —SO₂NR₁₁R₁₂.

5. A method according to claim 4 comprising administering to a patient in need thereof a therapeutically effective amount of at least one compound, or a pharmaceutically-acceptable salt, prodrug, stereoismer or solvate thereof, in which:
R₁₀ is hydrogen, C₁₋₆alkyl, substituted C₁₋₆alkyl, aryl or heteroaryl;
R₁₁ and R₁₂ are selected from hydrogen, C₁₋₆alkyl and substituted C₁₋₆alkyl;

6. A method according to claim 5 comprising administering to a patient in need thereof a therapeutically effective amount of at least one compound, or a pharmaceutically-acceptable salt, prodrug, stereoismer or solvate thereof, wherein:
the substituted C₁₋₆alkyl of groups R₁₀, R₁₁ and R₁₂ is substituted by one or more groups selected from
(a) —C(═O)R₁₄, —CO₂R₁₄, —NR₁₅R₁₆; or
(b) halogen, nitro, cyano, alkyl, aryl, heterocyclo or heteroaryl, each group of which may optionally be further substituted by C₁₋₆alkyl, C₁₋₆haloalkyl, C₁₋₆alkyloxy and C₁₋₆haloalkyloxy; and
R₁₄, R₁₅, and R₁₆ are independently selected from hydrogen, alkyl, substituted alkyl, aryl, heteroaryl, cycloalkyl and heterocyclo, each group of which may optionally be further substituted by halogen, nitro, cyano, C₁₋₆alkyl, C₁₋₆haloalkyl, C₁₋₆alkyloxy, and C₁₋₆haloalkyloxy.

7. A method according to claim 6, comprising administering to a patient in need thereof a therapeutically effective amount of at least one compound, or a pharmaceutically-acceptable salt, prodrug, stereoismer or solvate thereof, in which:
R₁₄ is C₁₋₆alkyl; and
R₁₅, and R₁₆ are selected from hydrogen and C₁₋₆alkyl.

8. A method according to claim 1, comprising administering to a patient in need thereof a therapeutically effective amount of at least one compound, or a pharmaceutically-acceptable salt, prodrug, stereoismer or solvate thereof, in which A is phenyl.

9. A compound having the formula (Ia), 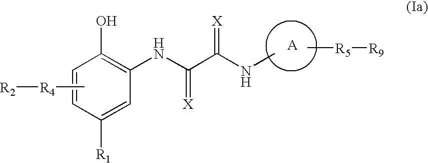
or a pharmaceutically-acceptable salt, prodrug, stereoisomer or solvate thereof, wherein:
A is aryl or heteroaryl;
X is O or S:
R₁ is halogen;
R₂-R₄ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
R₅ is —OR₁₇ or —SR₁₇;
R₆-R₉ are independently selected from hydrogen, alkyl, substituted alkyl, halogen, nitro, cyano, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀ and —N₁₃C(═O)NR₁₁R₁₂;
or any two of R₆-R₉ located on neighboring carbon atoms of the phenyl ring may be taken together to form a fused ring system in combination with the phenyl ring, wherein the fused ring system may be optionally further substituted; and
R₁₇ is hydrogen, alkyl, substituted alkyl, cycloalkyl, aryl heteroaryl or heterocyclo.

10. A compound according to claim 9, or a pharmaceutically-acceptable salt, prodrug, stereoismer or solvate thereof, in which R₁₇ is hydrogen or C₁₋₆alkyl.

11. A compound according to claim 9 or a pharmaceutically acceptable salt, prodrug, stereoismer or solvate thereof, in which R₂—R₄ and R₆-R₉ are independently selected from hydrogen, alkyl, substituted alkyl, halogen, nitro, cyano, —OR₁₀, —OC(═O)R₁₀, —CO₂R₁₀, —C(═O)NR₁₁R₁₂, —NR₁₁R₁₂, —S(═O)R₁₀, —SO₂R₁₀, and —SO₂NR₁₁R₁₂.

12. A compound according to claim 11, or a pharmaceutically acceptable salt, prodrug or solvate thereof, in which:
R₁₀ is hydrogen, C₁₋₆alkyl, substituted C₁₋₆alkyl, aryl or heteroaryl;
R₁₁ and R₁₂ are selected from hydrogen, C₁₋₆alkyl and substituted C₁₋₆alkyl;

13. A compound according to claim 10, or a pharmaceutically acceptable salt, prodrug, stereoismer or solvate thereof, wherein:
the substituted C₁₋₆alkyl of groups R₁₀, R₁₁ and R₁₂ is substituted by one or more groups selected from
(a) —C(═O)R₁₄, —CO₂R₁₄, —NR₁₅R₁₆; or
(b) alkyl, aryl, heterocyclo or heteroaryl, each group of which may optionally be further substituted by C₁₋₆alkyl, C₁₋₆alkyloxy, C₁₋₆haloalkyloxy, halogen, nitro and cyano; and
R₁₄, R₁₅, and R₁₆ are independently selected from hydrogen, alkyl, substituted alkyl, aryl, heteroaryl, cycloalkyl and heterocyclo, each group of which may optionally be further substituted by halogen, nitro, cyano, C₁₋₆alkyl, C₁₋₆haloalkyl, C₁₋₆alkyloxy, and C₁₋₆haloalkyloxy.

14. A compound according to claim 11, or a pharmaceutically acceptable salt, prodrug, stereoismer or solvate thereof, wherein:
R₁₄ is C₁₋₆alkyl; and
R₁₅, and R₁₆ are selected from hydrogen and C₁₋₆alkyl.

15. A compound according to claim 9 or a pharmaceutically-acceptable salt, prodrug, stereoisomer or solvate thereof, having the formula: (Ib), 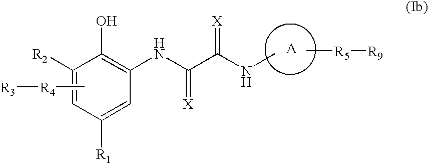
wherein R₂ is a halogen.

16. A compound according to claim 9, or a pharmaceutically-acceptable salt, prodrug, stereoismer or solvate thereof, in which A is phenyl.

17. A compound selected from
(i) N,N′-Bis-(3,5-dichloro-2-hydroxy-phenyl)-oxalamide;
N-(3,5-Dichloro-2-hydroxy-phenyl)-N′-(2-hydroxy-phenyl)-oxalamide;
N-(3,5-Dichloro-2-hydroxy-phenyl)-N′-(2-hydroxy-4-methyl-phenyl)-oxalamide;
N-(3,5-Dichloro-2-hydroxy-phenyl)-N′-(2-hydroxy-5-nitro-phenyl)-oxalamide;
N-(3,5-Dichloro-2-hydroxy-phenyl)-N′-(4-hydroxy-biphenyl-3-yl)-oxalamide;
N-(3,5-Dichloro-2-hydroxy-phenyl)-N′-(3-hydroxy-naphthalen-2-yl)-oxalamide;
N-(5-Chloro-2-hydroxy-phenyl)-N′-(3,5-dichloro-2-hydroxy-phenyl)-oxalamide;
N-(3,5-Dichloro-2-hydroxy-phenyl)-N′-(2-hydroxy-naphthalen-1-yl)-oxalamide;
N-(3,5-Dichloro-2-hydroxy-phenyl)-N′-[4-(1,1-dimethyl-propyl)-2-hydroxy-phenyl]-oxalamide;
N-(3,5-Dichloro-2-hydroxy-phenyl)-N′-(2-hydroxy-6-methyl-phenyl)-oxalamide;
N-(5-Chloro-2-hydroxy-4-nitro-phenyl)-N′-(3,5-dichloro-2-hydroxy-phenyl)-oxalamide;
4-[(3,5-Dichloro-2-hydroxy-phenylaminooxalyl)-amino]-3-hydroxy-benzoic acid methyl ester;
N-(3,5-Dichloro-2-hydroxy-4-methyl-phenyl)-N′-(3,5-dichloro-2-hydroxy-phenyl)-oxalamide;
N-(3,5-Dichloro-2-hydroxy-phenyl)-N′-(5-ethanesulfonyl-2-hydroxy-phenyl)-oxalamide;
N-(5-Cyano-2-hydroxy-phenyl)-N′-(3,5-dichloro-2-hydroxy-phenyl)-oxalamide;
N-(3,5-Dichloro-2-hydroxy-phenyl)-N′-(2-hydroxy-4-nitro-phenyl)-oxalamide;
N-(3,5-Dichloro-2-hydroxy-phenyl)-N′-(2-hydroxy-5-methoxy-phenyl)-oxalamide;
N-(3,5-Dichloro-2-hydroxy-phenyl)-N′-(5-fluoro-2-hydroxy-phenyl)-oxalamide;
N-(3-Chloro-2-hydroxy-phenyl)-N′-(3,5-dichloro-2-hydroxy-phenyl)-oxalamide;
N-(3,5-Dichloro-2-hydroxy-phenyl)-N′-(3,4-difluoro-2-hydroxy-phenyl)-oxalamide;
N-(3,5-Dichloro-2-hydroxy-phenyl)-N′-(2-hydroxy-5-trifluoromethyl-phenyl)-oxalamide;
N-(3,5-Dichloro-2-hydroxy-phenyl)-N′-(2-hydroxy-5-sulfamoyl-phenyl)-oxalamide;
3-[(3,5-Dichloro-2-hydroxy-phenylaminooxalyl)-amino]-4-hydroxy-benzoic acid;
4-[(3,5-Dichloro-2-hydroxy-phenylaminooxalyl)-amino]-3-hydroxy-benzoic acid;
3-[(3,5-Dichloro-2-hydroxy-phenylaminooxalyl)-amino]-4-hydroxy-benzoic acid ethyl ester;
N-(3,5-Dichloro-2-hydroxy-phenyl)-N′-(3-fluoro-2-hydroxy-phenyl)-oxalamide;
N-(3,5-Dichloro-2-hydroxy-phenyl)-N′-(2,3,5-trichloro-6-hydroxy-phenyl)-oxalamide;
N,N′-Bis-(3,5-dichloro-2-hydroxy-4-methyl-phenyl)-oxalamide;
N-(3,5-Dichloro-2-hydroxy-phenyl)-N′-(2-hydroxy-5-phenethylcarbamoyl-phenyl)-oxalamide;
N-(3,5-Dichloro-2-hydroxy-phenyl)-N′-{2-hydroxy-5-[3-(4-methyl-piperazin-1-yl)-propylcarbamoyl]-phenyl}-oxalamide;
{3-[(3,5-Dichloro-2-hydroxy-phenylaminooxalyl)-amino]-4-hydroxy-benzoylamino}-acetic acid ethyl ester;
2(2R)-{3-[(3,5-Dichloro-2-hydroxy-phenylaminooxalyl)-amino]-4-hydroxy-benzoylamino}-3-phenyl-propionic acid ethyl ester;
N-(3,5-Dichloro-2-hydroxy-phenyl)-N′-{2-hydroxy-5-[(pyridin-4-ylmethyl)-carbamoyl]-phenyl}-oxalamide;
N-(3,5-Dichloro-2-hydroxy-phenyl)-N′-{2-hydroxy-5-[2-(1-methyl-pyrrolidin-2-yl)-ethylcarbamoyl]-phenyl}-oxalamide (racemic);
N-(3,5-Dichloro-2-hydroxy-phenyl)-N′-[5-(3-dimethylamino-propylcarbamoyl)-2-hydroxy-phenyl]-oxalamide;
N-(3,5-Dichloro-2-hydroxy-phenyl)-N′-[2-hydroxy-5-(3-imidazol-1-yl-propylcarbamoyl)-phenyl]-oxalamide;
Hydrochloride of N-(3,5-Dichloro-2-hydroxy-phenyl)-N′-[5-(2-dimethylamino-ethylcarbamoyl)-2-hydroxy-phenyl]-oxalamide;
Hydrochloride of N-(3,5-Dichloro-2-hydroxy-phenyl)-N′-[5-(2-dimethylamino-ethylcarbamoyl)-2-hydroxy-phenyl]-oxalamide;
Hydrochloride of N-(3,5-Dichloro-2-hydroxy-phenyl)-N′-{2-hydroxy-5-[(pyridin-2-ylmethyl)-carbamoyl]-phenyl}-oxalamide;
{3-[(3,5-Dichloro-2-hydroxy-phenylaminooxalyl)-amino]-4-hydroxy-benzoylamino}-acetic acid;
2(2R)-{3-[(3,5-Dichloro-2-hydroxy-phenylaminooxalyl)-amino]-4-hydroxy-benzoylamino}-3-phenyl-propionic acid;
N-(3,5-Dichloro-2-hydroxy-phenyl)-N′-[2-hydroxy-4-(3-morpholin-4-yl-propylcarbamoyl)-phenyl]-oxalamide;
N-(3,5-Dichloro-2-hydroxy-phenyl)-N′-[4-(3-dimethylamino-propylcarbamoyl)-2-hydroxy-phenyl]-oxalamide;
N-(3,5-Dichloro-2-hydroxy-phenyl)-N′-[2-hydroxy-4-(3-imidazol-1-yl-propylcarbamoyl)-phenyl]-oxalamide;
N-(5-Amino-2-hydroxy-phenyl)-N′-(3,5-dichloro-2-hydroxy-phenyl)-oxalamide;
N,N′-Bis-(5-chloro-2-hydroxy-4-nitro-phenyl)-oxalamide;
N,N′-Bis-(4-amino-5-chloro-2-hydroxy-phenyl)-oxalamide dihydrochloride;
2-[(3,5-Dichloro-2-hydroxy-phenylaminooxalyl)-amino]-4-methyl-thiophene-3-carboxylic acid ethyl ester; and
N-(3,5-Dichloro-2-hydroxy-phenyl)-N′-(3,5-dichloro-pyridin-2-yl)-oxalamide; or
(ii) a pharmaceutically acceptable salt, prodrug, stereoisomer or solvate of (i) thereof.

18. A pharmaceutical composition, comprising: a pharmaceutically acceptable carrier and a therapeutically effective amount of a compound of claim 9.

19. A method according to claim 1, wherein the disorder associated with high levels of PAI-1 is a thromboembolic disorder.

20. A method according to claim 19, wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

21. A method for treating a thromboembolic disorder, comprising: administering to a patient in need thereof a therapeutically effective amount of a first and second therapeutic agent, wherein the first therapeutic agent is a compound having formula (I) or a pharmaceutically acceptable salt or hydrate thereof and the second therapeutic agent is at least one agent selected from a second PAI-1 inhibitor, a factor Xa inhibitor, an anti-coagulant agent, an anti-platelet agent, a thrombin inhibiting agent, a thrombolytic agent, and a fibrinolytic agent wherein formula (I) is 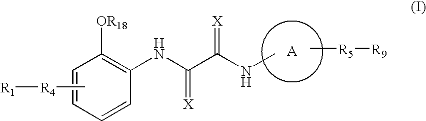
or a pharmaceutically-acceptable salt, prodrug, stereoismer or solvate thereof, wherein:
A is aryl or heteroaryl;
X is O or S;
R₁-R₉ are independently selected from hydrogen, alkyl, substituted alkyl, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀, —N₁₃C(═O)NR₁₁R₁₂, halogen, nitro and cyano;
or any two of R₁-R₉ located on neighboring carbon atoms of the phenyl ring may be taken together to form a fused ring system in combination with the phenyl ring to which they are attached, wherein the fused ring system may be optionally further substituted;
R₁₀, R₁₁, R₁₂ and R₁₃ are independently selected from hydrogen, alkyl, substituted alkyl, aryl, heteroaryl, cycloalkyl and heterocyclo, wherein each instance of R₁₀, R₁₁, R₁₂ and/or R₁₃ is selected independently; and
R₁₈ is hydrogen, alkyl or substituted alkyl.

22. A method according to claim 21 wherein the second therapeutic agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

23. The method according to claim 22, wherein the second therapeutic agent is at least one anti-platelet agent.

24. The method according to claim 23, wherein the anti-platelet agent is aspirin or clopidogrel.

25. The method according to claim 24, wherein the anti-platelet agent is clopidogrel.

26. An article of manufacture, comprising:
(a) a first container;
(b) a pharmaceutical composition located within the first container, wherein the composition, comprises: a first therapeutic agent, comprising: a compound of any one of formula (I), or a pharmaceutically acceptable salt or hydrate form thereof; and
(c) a package insert stating that the pharmaceutical composition can be used for the treatment of a thromboembolic disorder
wherein formula (I) is 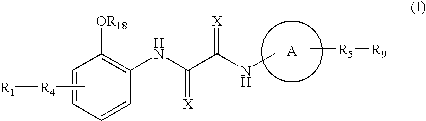
or a pharmaceutically-acceptable salt, prodrug, stereoismer or solvate thereof, wherein:
A is aryl or heteroaryl;
X is O or S;
R₁-R₉ are independently selected from hydrogen, alkyl, substituted alkyl, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀, —N₁₃C(═O)NR₁₁R₁₂, halogen, nitro and cyano;
or any two of R₁-R₉ located on neighboring carbon atoms of the phenyl ring may be taken together to form a fused ring system in combination with the phenyl ring to which they are attached, wherein the fused ring system may be optionally further substituted;
R₁₀, R₁₁, R₁₂ and R₁₃ are independently selected from hydrogen, alkyl, substituted alkyl, aryl, heteroaryl, cycloalkyl and heterocyclo, wherein each instance of R₁₀, R₁₁, R₁₂ and/or R₁₃ is selected independently; and
R₁₈ is hydrogen, alkyl or substituted alkyl.

27. An article of manufacture according to claim 26, further comprising:
(d) a second container;
wherein components (a) and (b) are located within the second container and component (c) is located within or outside of the second container.

28. A compound of claim 8, or a pharmaceutically acceptable salt or hydrate form thereof, for use in therapy.

29. Use of a compound having formula (I), for the manufacture of a medicament for the treatment of a thromboembolic disorder wherein formula (I) is 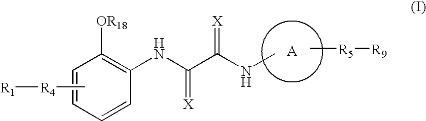
or a pharmaceutically-acceptable salt, prodrug, stereoismer or solvate thereof, wherein:
A is aryl or heteroaryl;
X is O or S;
R₁-R₉ are independently selected from hydrogen, alkyl, substituted alkyl, —OR₁₀, —SR₁₀, —OC(═O)R₁₀, —CO₂R₁₀, —C(═O)NR₁₁R₁₂, —NR₁₁R₁₂, —S(═O)R₁₀, —SO₂R₁₀, —SO₂NR₁₁R₁₂, —NR₁₃SO₂NR₁₁R₁₂, —NR₁₃SO₂R₁₀, —NR₁₃C(═O)R₁₀, —NR₁₃CO₂R₁₀, —N₁₃C(═O)NR₁₁R₁₂, halogen, nitro and cyano;
or any two of R₁-R₉ located on neighboring carbon atoms of the phenyl ring may be taken together to form a fused ring system in combination with the phenyl ring to which they are attached, wherein the fused ring system may be optionally further substituted;
R₁₀, R₁₁, R₁₂ and R₁₃ are independently selected from hydrogen, alkyl, substituted alkyl, aryl, heteroaryl, cycloalkyl and heterocyclo, wherein each instance of R₁₀, R₁₁, R₁₂ and/or R₁₃ is selected independently; and
R₁₈ is hydrogen, alkyl or substituted alkyl.
